SEQUENCE LISTING (6 sequences)

SEQ ID NO: 1 - DNA, 20 bp, synthetic construct
  source 1..20: mol_type = other DNA
tctttagcatctgccgggtg

SEQ ID NO: 2 - DNA, 20 bp, synthetic construct
  source 1..20: mol_type = other DNA
gagccaatggagcttaggca

SEQ ID NO: 3 - DNA, 20 bp, synthetic construct
  source 1..20: mol_type = other DNA
cttacggaagcacccacgat

SEQ ID NO: 4 - DNA, 20 bp, synthetic construct
  source 1..20: mol_type = other DNA
cgtctccacggaaacagcat

SEQ ID NO: 5 - DNA, 24 bp, synthetic construct
  source 1..24: mol_type = other DNA
gcctagttattgatgatccagggt

SEQ ID NO: 6 - DNA, 23 bp, synthetic construct
  source 1..23: mol_type = other DNA
agagacttgaatttgccctaaca

(19) United States
(12) Patent Application Publication
(10) Pub. No.: US 2024/0173401 A1
(43) Pub. Date: May 30, 2024

(54) Compositions and Methods to Enhance Therapeutic Efficacy of Cancer Therapies

(71) Applicant: Wisconsin Alumni Research Foundation, Madison, WI (US)

(72) Inventors: Zachary S. Morris, Madison, WI (US); Justin C. Jagodinsky, Madison, WI (US)

(21) Appl. No.: 18/471,422

(22) Filed: Sep. 21, 2023

Related U.S. Application Data
(60) Provisional application No. 63/408,610, filed on Sep. 21, 2022.

(51) Int. Cl. A61K 39/39; A61K 39/395; A61N 5/10; A61P 35/00; A61P 37/04

(52) U.S. Cl. CPC A61K 39/39; A61K 39/3955; A61N 5/10; A61P 35/00; A61P 37/04; A61K 2039/55572

(57) ABSTRACT

This disclosure relates to compositions and methods to enhance therapeutic efficacy of cancer therapies.

### CROSS REFERENCE TO RELATED APPLICATIONS

This application claims the benefit of U.S. Provisional Application No. 63/408,610, filed Sep. 21, 2022, which is incorporated by reference herein in its entirety.

### INCORPORATION BY REFERENCE OF SEQUENCE LISTING PROVIDED ELECTRONICALLY

This application contains a Sequence Listing submitted as an electronic text file named “22-0925-WO_ST26.xml,” having a size in bytes of 6,159 bytes, and created on Sep. 20, 2023. The information contained in this electronic file is hereby incorporated by reference in its entirety.

### BACKGROUND OF THE DISCLOSURE

### Field of Invention

This disclosure relates to compositions and methods to enhance therapeutic efficacy of cancer therapies.

### Technical Background

Radiation therapy (RT) has been demonstrated to generate an in situ vaccination (ISV) effect in murine models and in cancer patients by triggering the death of tumor cells in a way that they present tumor antigens to immune cells. Unfortunately, radiation therapy also has negative effects on the tumor microenvironment that can inhibit immune cell activity against the tumor which is why radiation therapy generally does not induce systemic immune activation and subsequent generation of out of field anti-tumor responses (i.e., abscopal responses) remains exceedingly rare when radiation therapy is used. Checkpoint inhibitors are an emerging clinical class of molecules that block checkpoint pathways in immune cells leading to immune stimulation. The release of antigens from the tumor upon radiation added to the inhibition of the checkpoint pathways to stimulate immune cells should provide an enhanced tumor killing response. However, radiation has not routinely translated into enhanced clinical response to immune checkpoint inhibition (ICI). Therefore, there is a need for additional treatments to further stimulate the immune system to enhance the anti-cancer activity of radiation in combination with checkpoint inhibitors.

### SUMMARY OF THE DISCLOSURE

This disclosure provides compositions and methods to enhance therapeutic efficacy of cancer therapies.

In a first aspect, the present disclosure provides a method of treating cancer in a subject in need thereof. The method includes the steps of administering a therapeutically effective amount of an adjuvant to a tumor of the subject, wherein the adjuvant is administered intratumorally, administering a therapeutically effective amount of a radiotherapy and/or local ablative therapy to the tumor, administering a therapeutically effective amount of an immune checkpoint inhibitor to the subject, and inducing an immune response to the cancer.

In one embodiment of the first aspect, the adjuvant comprises a TLR4 agonist. In one embodiment of the first aspect, the TLR4 agonist comprises one or more of monophosphoryl lipid A, monophosphoryl lipid A-504, monophosphoryl tri-acyl lipid A, monophosphoryl 3-deacyl lipid A, monophosphoryl tetra-acyl lipid A, monophosphoryl hexa-acyl lipid A, 3-deacyl, D-(+)-trehalose 6,6′-dibehenate, and dimethyldioctadecylammonium (bromide salt). In one embodiment of the first aspect, the therapeutically effective amount of the adjuvant comprises about 20 μg to about 70 mg or about 0.5 to about 5 mg/kg. In one embodiment of the first aspect, the radiotherapy comprises external beam radiation therapy (EBRT) and/or internal radiation therapy. In one embodiment of the first aspect, the radiotherapy is EBRT. In one embodiment of the first aspect, the internal radiation therapy comprises brachytherapy and/or radiopharmaceutical. In one embodiment of the first aspect, the therapeutically effective amount of radiotherapy is about 2 to about 20 Gy. In one embodiment of the first aspect, the therapeutically effective amount of radiotherapy is administered in a gradient dose of about 2 Gy/min. In one embodiment of the first aspect, the local ablative therapy comprises radiofrequency ablation, microwave ablation, and/or cryoablation. In one embodiment of the first aspect, the immune checkpoint inhibitor comprises one or more therapeutic agents that inhibit CTLA-4, PD-1, and/or PD-L1. In one embodiment of the first aspect, the immune checkpoint inhibitor comprises an anti-CTLA-4 antibody.

In a second aspect, the present disclosure provides a composition for treating cancer. The composition includes a therapeutically effective amount of an adjuvant, a therapeutically effective amount of an immune checkpoint inhibitor, and a pharmaceutically acceptable carrier or diluent,

In one embodiment of the second aspect, the adjuvant comprises a TLR4 agonist. In one embodiment of the second aspect, the TLR4 agonist comprises one or more of monophosphoryl lipid A, monophosphoryl lipid A-504, monophosphoryl tri-acyl lipid A, monophosphoryl 3-deacyl lipid A, monophosphoryl tetra-acyl lipid A, monophosphoryl hexa-acyl lipid A, 3-deacyl, D-(+)-trehalose 6,6′-dibehenate, and dimethyldioctadecylammonium (bromide salt). In one embodiment of the second aspect, the immune checkpoint inhibitor comprises one or more therapeutic agents that inhibit CTLA-4, PD-1, and/or PD-L1. In one embodiment of the second aspect, the immune checkpoint inhibitor comprises an anti-CTLA-4 antibody.

In a second aspect, the present disclosure provides a method of treating cancer in a subject in need thereof. The method includes the steps of administering a therapeutically effective amount of a TLR4 agonist to a tumor of the subject, wherein the TLR4 agonist is administered intratumorally, administering a therapeutically effective amount of EBRT to the tumor, administering a therapeutically effective amount of an anti-CTLA-4 antibody to the subject, and at least one of reducing tumor volume, increasing overall survival of the subject, and increasing complete response rate in the subject.

In one embodiment of the third aspect, the TLR4 agonist comprises one or more of monophosphoryl lipid A, monophosphoryl lipid A-504, monophosphoryl tri-acyl lipid A, monophosphoryl 3-deacyl lipid A, monophosphoryl tetra-acyl lipid A, monophosphoryl hexa-acyl lipid A, 3-deacyl, D-(+)-trehalose 6,6′-dibehenate, and dimethyldioctadecylammonium (bromide salt). In one embodiment of the third aspect, the therapeutically effective amount of the TLR4 agonist comprises about 0.5 to about 5 mg/kg. In one embodiment of the third aspect, the therapeutically effective amount of EBRT is about 2 to about 20 Gy administered in a gradient dose of about 2 Gy/min. In one embodiment of the third aspect, the therapeutically effective amount of the anti-CTLA-4 antibody is about 10 mg/kg.

In one embodiment according to either the first or third aspects or embodiments thereof, the cancer comprises one or more solid tumors. In one embodiment, the cancer is melanoma or prostate cancer.

In one embodiment according to either the first or third aspects or embodiments thereof, the methods further includes the step of increasing production of Th1-associated, IgG2c anti-tumor antibodies associated with the tumor.

In one embodiment according to either the first or third aspects or embodiments thereof, the methods further includes the step of inducing a systemic anti-tumor immune response.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure. All data presented are reported as mean±SEM unless otherwise noted. For all graphs, *, p<0.05; * *, p<0.01; * * *, p<0.001; and ****, p<0.0001.

FIGS. 1A-1K. MPL enhances efficacy of RT+C4 in B78 melanoma and Myc-CaP prostate cancer models. (1A) Mice with a single B78 or Myc-CaP flank tumor were treated with PBS (p<0.05 vs. RT+C4 and RT+C4+MPL), External Beam Radiation (RT, 12 Gy; p<0.05 vs. RT+C4+MPL), RT+anti-CTLA-4 (C4, 200 μg; p<0.05 vs. PBS, RT, and RT+C4+MPL), RT+C4+MPL (MPL, 20 μg; p<0.05 vs. PBS, RT, RT+C4, and MPL), or MPL alone (p<0.05 vs. RT+C4 and RT+C4+MPL). Tumor response, by group, by individual animal, and animal survival are shown for B78 (in 1B, 1C, and 1D (PBS-p<0.05 vs. RT+C4 and RT+C4+MPL; RT-p<0.05 vs. RT+C4+MPL, RT+C4-p<0.05 vs. PBS, RT, and RT+C4+MPL; RT+C4+MPL-p<0.05 vs. PBS, RT, RT+C4, and MPL, MPL-p<0.05 vs. RT+C4 and RT+C4+MPL)). Mice with complete response to treatment with either RT+C4 or RT+C4+MPL (1E) were rechallenged with the same tumor they initially rejected (1F). Tumor response, by group, by individual animal, and animal survival are shown for Myc-CaP (in 1G (PBS-p<0.05 vs. RT+C4+MPL; RT-p<0.05 vs. RT+C4+MPL, RT+C4 p<0.05 vs. RT+C4+MPL; RT+C4+MPL-p<0.05 vs. PBS, RT, RT+C4, and MPL, MPL-p<0.05 vs. RT+C4+MPL), 1H, and 1I (PBS-p<0.05 vs. RT+C4 and RT+C4+MPL; RT-p<0.05 vs. RT+C4 and RT+C4+MPL, RT+C4-p<0.05 vs. PBS, RT, RT+C4+MPL, and MPL; RT+C4+MPL-p<0.05 vs. PBS, RT, RT+C4, and MPL, MPL-p<0.05 vs. RT+C4 and RT+C4+MPL)). Mice with complete response to treatment with either RT+C4 or RT+C4+MPL (1J) were rechallenged with the same tumor they initially rejected (1K). N=10-16 mice per group. Significance determined by linear mixed effects regression analysis with Tukey multiple comparisons testing for tumor growth (significant differences, p<0.05, demarcated by * with the color of the asterisk representing which group from which the sample is significantly different), Kaplan-Meier estimation with log-rank testing and Cox regression for survival analysis (significant differences, p<0.05, demarcated by * with the color of the asterisk representing which group from which the sample is significantly different), and Chi squared test for complete response rate. MPL, monophosphoryl lipid; RT, radiation therapy; EBRT, external beam radiation therapy; PBS, phosphate buffered saline.

FIGS. 2A-2I. MPL promotes Th1 antibody class switching and correlates with depth of tumor response. To determine the presence of anti-tumor antibodies serum was isolated from mice bearing B78 tumors at day 15 and 30 following treatment initiation. Serum was incubated with B78 cells and antibody class was determined using secondary antibodies against IgG, IgG1, and IgG2c (2A). Ratio of IgG2c:IgG1 at D15 was increased in RT+C4+MPL compared to other groups without a change in overall IgG (2B, 2C). Similar trends in IgG2c:IgG1 ratio were observed at day 30 however did not reach statistical significance (2D, 2E). In a treatment agnostic fashion, mice were reclassified based on depth of response using RECIST criteria 1.1 [PD: progressive disease N=33, SD: stable disease N=6, PR: partial response N=7, CR: complete response N=9] and ratio of IgG2c:IgG1 was quantified (2F). Mice experiencing any response (SD, PR, or CR, N=22) were also pooled and the IgG2c:IgG1 ratio was compared to nonresponding mice (PD, N=33) (2G). Within treatment groups that had mice experiencing a complete response (RT+C4 and RT+C4+MPL) the IgG2c:IgG1 was quantified in mice experiencing a complete response (RT+C4, N=2 and RT+C4+MPL, N=7) and was compared to nonresponding mice (RT+C4, N=14 and RT+C4+MPL, N=9). The IgG2c:IgG1 ratio was elevated in complete responding mice treated with RT+C4+MPL (2H) but not RT+C4 (2I). One-way ANOVA with Tukey's honestly significant difference (HSD) test to adjust for multiple comparisons was used to assess statistical significance of observed mean differences in IgG2c:IgG1 ratio. For comparisons between two groups a Student's t-test was performed.

FIGS. 3A-30. MPL reprograms the immune microenvironment to favor M1 and Th1 polarization. Flow cytometry analyses of tumor immune cell infiltrates [total macrophages (CD11b+F4/80+), M1 macrophages (CD80+), M2 macrophages (CD206+), ratio of M1:M2 macrophages, and CD8+ T cells (CD8+)] (3A-3E) and lymph node immune populations [classical dendritic cells (CD11c+CD103+MHC-II+CD80+), Th1 cells (TBET+CD4+), resident memory CD4 T cells (CD4+CD103+), Ifnγ producing CD8 T cells (CD8+Ifnγ+), and resident memory CD8 T cells (CD8+CD103+)] (3F-3J) as a percent of total live cells is shown at day 15 following treatment initiation in B78 melanoma. N=7 mice per group. (3K) Tumors from a separate cohort of mice were subjected to cytokine profiling. Cytokine and chemokine concentrations in tumor lysates were measured by multiplex immunoassay. Hierarchical clustering analysis was performed, and the assay results were displayed as a Z-score for each cytokine or chemokine. The addition of MPL to RT+C4 increases expression of TLR4 associated cytokines (3L), increases Th1 cytokines (3M), decreases Th2 cytokines (3N), and increases several pro-inflammatory M1 cytokines compared to RT+C4 (3O). N=6 mice per group. CR, complete response; MFI, median fluorescence intensity; MPL, monophosphoryl lipid; RT, radiation therapy; TBET, T-box transcription factor 21.

FIGS. 4A-40. MPL directly activates macrophages to influence polarization of Th cells. Bone marrow derived macrophages were cultured in the presence of increasing amounts of MPL (0, 5, 20, 100, 500 ng/mL) and 24 hours later were harvested for qPCR analysis to quantify polarization (4A), pro-inflammatory cytokines (4B), and anti-inflammatory markers (4C). This was repeated with freshly isolated CD4 and CD8 cells from mouse spleens to determine CD4 polarization (4D), CD4 activation (4E), and CD8 activation (4F) following MPL treatment (for 4A-4F, data corresponding to concentrations of 5, 20, 100, and 500 ng/mL MPL are presented left to right for each gene measured). The potential for MPL stimulated macrophages to activate CD8 T cells and polarize CD4 T cells was determined using a co-culture system (4G). MPL stimulated macrophages increase activation of CD8 T cells (CD69+) when cocultured (4H), but not when in the presence of CD4 T cells (4I). MPL stimulated macrophages increase Th1 polarization (CXCR3+) (4J), decrease Th2 polarization (CXCR4+) (4K), and increase regulatory T cell polarization (CD25+FOXP3+) (4L) when co-cultured. Identical trends were observed with the addition of CD8 T cells (4M-4O). One way ANOVA with Tukey's honestly significant difference (HSD) test to adjust for multiple comparisons was used to assess statistical significance of observed mean differences in gene expression (significant differences, *p<0.05, **p<0.01 ***p<0.001, ****p<0.0001). N=5 replicates per group.

FIGS. 5A-5H. Serum antibody bound to tumor cells enhances MPL induced activation of macrophages. Bone marrow derived macrophages were radiated in culture (12 Gy) and media was immediately exchanged with fresh media containing 100 ng/mL MPL. After 24 hours cells were harvested for qPCR analysis of polarization and activation markers (5A). The capacity for serum derived anti-tumor antibodies to activate macrophages was tested using a co-culture system (5B). Macrophages were cultured with PBS, MPL (100 ng/mL), serum from mice rendered disease free of B78 tumors via RT+C4+MPL treatment, or both MPL and serum. After 24 hours cells were harvested for analysis of polarization (5C), and activation markers (5D). To test whether macrophages can be active in the presence of tumor cells, macrophages were cultured with or without 100 ng/mL MPL and 24 hours later B78 cells were added with or without serum from disease free mice. After 24 hours of co-culture macrophages were harvested for analysis of polarization (5E) and activation markers (5F). To confirm tumor specificity of the serum antibodies, this was repeated with the unrelated cell line Myc-CaP (5G, 5H). One-way ANOVA with Tukey's honestly significant difference (HSD) test to adjust for multiple comparisons was used to assess statistical significance of observed mean differences in gene expression (significant differences, *p<0.05, **p<0.01 ***p<0.001, ****p<0.0001). N=3 replicates per group.

FIGS. 6A-6F. Radiation downregulates TLR4 expression on macrophages and MHC-IT expression on B16 tumor cells and cytotoxic CD4 cells are not generated by combination therapy but B cells are modestly activated with radiation but not MPL or serum. Macrophages cultured in vitro were treated with PBS, anti-CTLA-4 (C4, 5 μg), radiation (RT, 12 Gy) or RT+C4 and expression of TLR4 was quantified using flow cytometry 24 hours following treatment (6A). B16 melanoma cells cultured in vitro were treated with PBS or radiation (RT, 12 Gy) and expression of MHC-II was quantified using flow cytometry 3 days following treatment (6B). B16 tumors were treated with PBS or RT+C4+MPL and expression of MHC-II was quantified using flow cytometry 15 days following treatment (6C). CD4 cells were also harvested and co-cultured with B16 cells that had received either 0 Gy or 12 Gy of RT 7 days prior to coculture and tumor cell killing was quantified using Annexin V staining (6D). B cells cultured in vitro were treated with either PBS, MPL (100 μg), serum from disease free mice, or MPL+serum and activation markers were quantified using qPCR (6E). B cells cultured in vitro were treated with either PBS, C4 (5 μg), RT (12 Gy), or RT+C4 and activation markers were quantified using qPCR (6F).

FIGS. 7A-7H. Serum antibody induced macrophage activation is dependent on Fcγ receptor and is critical for anti-tumor response. Significance of Fcγ receptor was tested in vivo. Wild type and Fcγ receptor deficient mice were treated with either PBS or RT+C4+MPL (3×), and tumor growth and survival was tracked (7A, 7B (PBS WT-p<0.05 vs. 3×WT and 3×WT-p<0.05 vs. PBS WT)). N=5 mice per group. Bone marrow derived macrophages were radiated in culture (12 Gy) and media was immediately exchanged with fresh media containing 100 ng/mL MPL. After 24 hours cells were harvested for qPCR analysis of Fcγ receptor expression (7C). To determine the importance of Fcγ receptor on macrophage activation, macrophages were cultured with PBS, MPL (100 ng/mL), serum from mice rendered disease free of B78 tumors via RT+C4+MPL treatment, or both MPL and serum. After 24 hours cells were harvested for analysis of polarization (7D), and activation markers (7E). To test whether macrophages can be active in the presence of tumor cells, macrophages were cultured with or without 100 ng/mL MPL and 24 hours later B78 cells were added with or without serum from disease free mice. After 24 hours of co-culture macrophages were harvested for analysis of polarization (7F) and activation markers (7G). N=3 replicates per group. To test whether serum derived anti-tumor antibodies can initiate ADCC (antibody-dependent cellular cytotoxicity), unstimulated and MPL stimulated macrophages were co-cultured with B78 cells with and without serum. After 24 hours cells were collected and analyzed via flow cytometry. CD45-Annexin V+cells are plotted (7H). N=5 replicates per group. One-way ANOVA with Tukey's honestly significant difference (HSD) test to adjust for multiple comparisons was used to assess statistical significance of observed mean differences in gene expression. Tumor growth was compared using linear mixed effects regression analysis with Tukey multiple comparisons testing. Kaplan-Meier estimation with log-rank testing and Cox regression were performed for survival analysis (significant differences, p<0.05, demarcated by * with the color of the asterisk representing which group from which the sample is significantly different).

FIGS. 8A-8H. MPL induced immune response is dependent on macrophages and Th1 CD4 T cells. Importance of different immune cell populations for the anti-tumor response generated via RT+C4+MPL was determined using antibody-based depletion. Mice bearing B78 tumors were treated with either PBS or RT+C4+MPL (3×) and either macrophages (αCD115), NK cells (αNK1.1), CD4 T cells (αCD4), CD8 T cells (αCD8), or both CD4 and CD8 T cells (αCD/CD8) were depleted, and tumor growth and survival was tracked (8A, 8B (PBS-p<0.05 vs. 3×+IgG, 3×+αNK1.1, and 3×+αCD8; 3×+IgG-p<0.05 vs. PBS, 3×+αCD115, 3×+αNK1.1, and 3×+αCD4/CD8; 3×+αCD115-p<0.05 vs. 3×+IgG, 3×+αNK1.1, and 3×+αCD8; 3×+αNK1.1-p<0.05 vs. PBS, 3×+αCD115, 3×+αCD4, and 3×+αCD4/CD8; 3×+αCD4-p<0.05 vs. 3×+IgG, 3×+αNK1.1, and 3×+αCD8; 3×+αCD8-p<0.05 vs. PBS, 3×+αCD115, 3×+αCD4, and 3×+αCD4/CD8; and 3×+αCD4/CD8-p<0.05 vs. 3×+IgG, 3×+αNK1.1, and 3×+αCD8)). N=5-10 mice per group. At day 15 following RT, serum was collected for antitumor antibody quantification (8C (PBS p<0.0001 vs. CD8, NK, and 3×; 3×-p<0.0001 vs. CD 115, CD4+CD8, CD4, and PBS; CD115-p<0.05 vs. CD8 and NK and p<0.0001 vs. CD4+CD8, CD4, and 3×; NK-p<0.05 vs. CD115 and p<0.0001 vs. CD4+CD8, CD4, and PBS; CD4-p<0.0001 vs. CD115, CD8, NK, and 3×; CD8-p<0.05 vs. CD115 and p<0.0001 vs. CD4+CD8, CD4, and PBS; and CD4+CD8-p<0.0001 vs. CD115, CD8, NK, and 3×), 8D (NK-p<0.05 vs. CD4+CD8 and p<0.01 vs. CD4; CD4-p<0.01 vs. CD8 and NK; CD8-p<0.01 vs. CD4+CD8 and CD4; and CD4+CD8-p<0.05 vs. NK and p<0.01 vs. CD8). Importance of Th1 CD4 T cells for the anti-tumor response generated via RT+C4+MPL was determined using TBET deficient mice. Wild type and TBET deficient mice (TBET KO) were treated with either PBS or RT+C4+MPL (3×), and tumor growth was tracked (8E (PBS WT-p<0.05 vs. 3×WT; and 3×WT-p<0.05 vs. PBS WT). N=5 mice per group. At day 15 following RT, serum was collected for anti-tumor antibody quantification (8F (3×WT-p<0.05 vs. 3×PBS WT and p <0.01 vs. PBS TBET KO and 3×TBET KO)) and tumor and draining lymph node were collected for infiltration analysis (8G, 8H). Tumor growth was compared using linear mixed effects regression analysis with Tukey multiple comparisons testing. Kaplan-Meier estimation with log-rank testing and Cox regression were performed for survival analysis (significant differences, p<0.05, demarcated by * with the color of the asterisk representing which group from which the sample is significantly different). One way ANOVA with Tukey's honestly significant difference (HSD) test to adjust for multiple comparisons was used to assess statistical significance of observed mean differences in lung metastases (significant differences, *, p<0.05; * *, p<0.01; * * *, p<0.001; and ****, p<0.0001, with color of asterisk representing which group from which the sample is significantly different).

FIGS. 9A-9C. MPL enhances systemic immune response independent of CD8 T cells. To determine whether CD8 T cells were required for generation of systemic immune response, mice bearing B78 primary tumors and B16 lung metastases (250,000 B16 cells injected via tail vein immediately following RT) were treated with either PBS, RT, RT+C4, RT+C4+MPL, or MPL. Additionally, a separate cohort of mice treated with RT+C4+MPL were also treated with CD8 depletion antibody. Tumor volume and survival were tracked (9A (PBS-p<0.05 vs. RT, RT+C4, RT+C4+MPL, and RT+C4+MPL+αCD8; RT-p<0.05 vs. PBS and RT+C4+MPL; RT+C4-p<0.05 vs. PBS and RT+C4+MPL; RT+C4+MPL-p<0.05 vs. PBS, RT, RT+C4, and MPL; RT+C4+MPL+αCD8-p<0.05 vs. PBS and MPL; and MPL-p<0.05 vs. RT+C4, RT+C4+MPL, and RT+C4+MPL+αCD8), 9B (PBS-p<0.05 vs. RT+C4+MPL and RT+C4+MPL+αCD8; RT-p<0.05 vs. PBS, RT+C4+MPL, and RT+C4+MPL+αCD8; RT+C4-p<0.05 vs. PBS and MPL; RT+C4+MPL-p<0.05 vs. PBS, RT, RT+C4, and MPL; RT+C4+MPL+αCD8-p<0.05 vs. PBS, RT, RT-C4, and MPL; and MPL-p<0.05 vs. RT+C4+MPL and RT+C4+MPL+αCD8). At time of death or day 60 following treatment initiation, lungs were removed and number of metastases were calculated (9C). N=5-6 mice per group. Tumor growth was compared using linear mixed effects regression analysis with Tukey multiple comparisons testing. Kaplan-Meier estimation with log-rank testing and Cox regression were performed for survival analysis (significant differences, p<0.05, demarcated by * with the color of the asterisk representing which group from which the sample is significantly different). One-way ANOVA with Tukey's honestly significant difference (HSD) test to adjust for multiple comparisons was used to assess statistical significance of observed mean differences in lung metastases (significant differences, *, p<0.05; **, p<0.01; * * *, p<0.001; and ****, p<0.0001, with color of asterisk representing which group from which the sample is significantly different).

FIGS. 10A-10D. Depletion confirmation. A multi-depletion study was performed to deplete T cells (10A), NK cells (10B), and macrophages (10C). A systemic disease study depleted CD8 T cells (10D).

### DETAILED DESCRIPTION

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

All publications, patents, and patent applications cited herein are hereby expressly incorporated by reference in their entirety for all purposes.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

In some embodiments, amounts or percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “₅%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

As used herein, the terms “therapeutic amount,” “therapeutically effective amount” or “effective amount” can be used interchangeably and refer an amount of a compound or material (i.e., a “therapeutic agent”) that becomes available through an appropriate route of administration to provide a therapeutic benefit to a patient for a disorder, a condition, or a disease. The amount of a compound which constitutes a “therapeutic amount,” “therapeutically effective amount” or “effective amount” will vary depending on the compound, the disorder and its severity, and the age of the subject to be treated, but can be determined routinely by one of ordinary skill in the art.

“Treating” or “treatment,” as used herein, covers the treatment of a disorder, condition, or a disease described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder. For example, the terms “treating,” “treat,” or “treatment” refer to either preventing development or exacerbation of, providing symptomatic relief for, or curing a patient's disorder, condition, or disease.

In view of the present disclosure, the methods and compositions described herein can be configured by the person of ordinary skill in the art to meet the desired need.

As used herein, the terms “patient,” “subject,” and “individual” can be used interchangeably and refer to an animal. For example, the patient, subject, or individual can be a mammal, such as a human to be treated for a disorder, condition, or a disease.

As used herein, the terms “disorder,” “condition,” and “disease” refer, for example, to cancer and its associated comorbidities.

It is noted that terms like “preferably,” “commonly,” and “typically” are not utilized herein to limit the scope of the methods and compositions as described herein or to imply that certain features are critical, essential, or even important to the structure or function of the subject matter recited in the claims.

In view of the present disclosure, the methods and compositions described herein can be configured by the person of ordinary skill in the art to meet the desired need.

Overview

Disclosed herein are methods of and combinatorial therapies for treating cancer that significantly reduce tumor growth, increase survival, and increase complete response rates in treated individuals. The present disclosure demonstrates the potential for adjuvants (e.g., vaccine adjuvants) to enhance the efficacy of in situ tumor vaccine approaches in combination with radiation therapy and checkpoint inhibitor treatment to drive the immune system's response to tumor cells.

Methods

In one embodiment, the present disclosure provides a method of treating cancer in a subject in need thereof that can include administering a therapeutically effective amount of an adjuvant to a tumor of the subject, wherein the adjuvant is administered intratumorally. The method can further includes administering a therapeutically effective amount of a radiotherapy to the tumor and administering a therapeutically effective amount of an immune checkpoint inhibitor to the subject. The method can lead to the induction of an immune response to the cancer that treats the subject's disease.

In some embodiments, the adjuvant is a vaccine adjuvant. In some embodiments, the adjuvant is a lipopolysaccharide or a derivative thereof. In some embodiments, the adjuvant is a lipopolysaccharide (LPS) component of the cell-wall of Salmonella entericais. In some embodiments, the adjuvant is a TLR4 agonist. Examples of TLR4 agonists contemplated for use herein include those having the structures shown below.

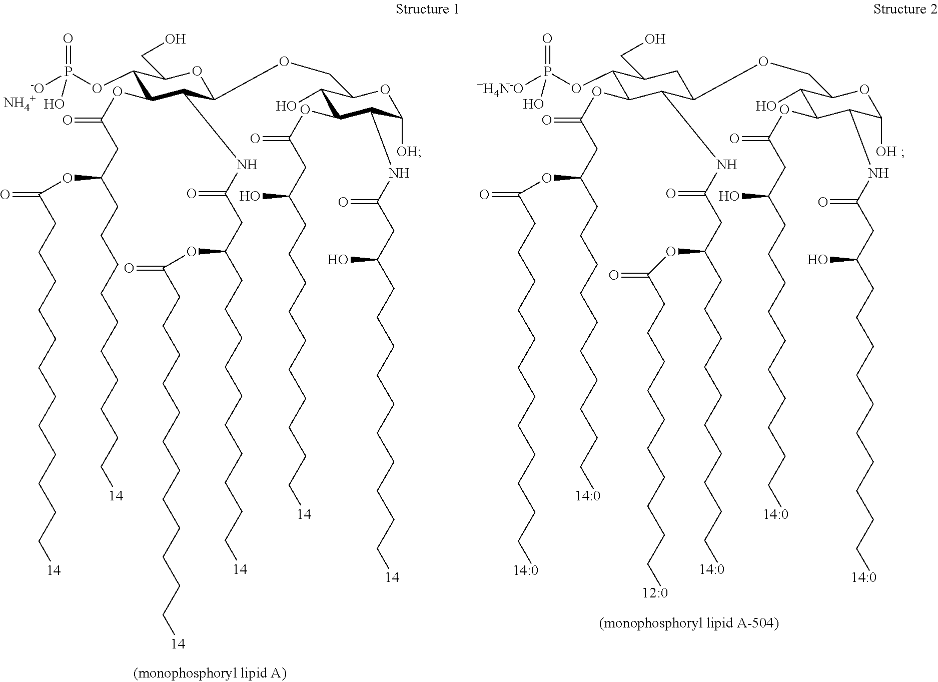

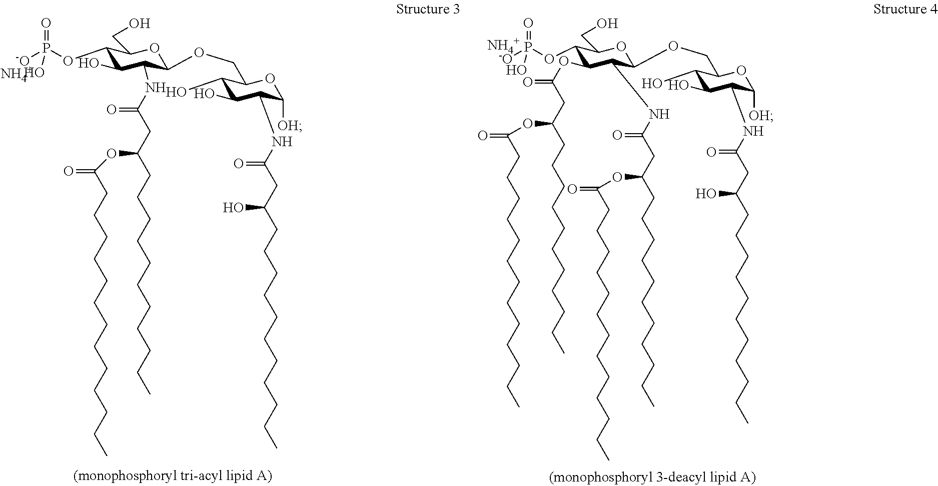

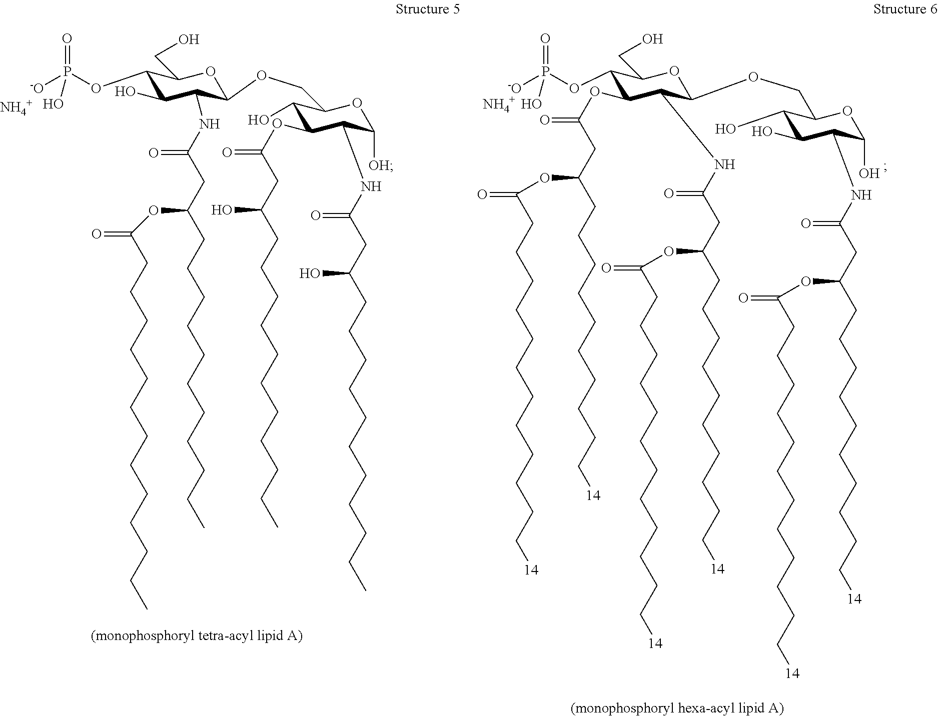

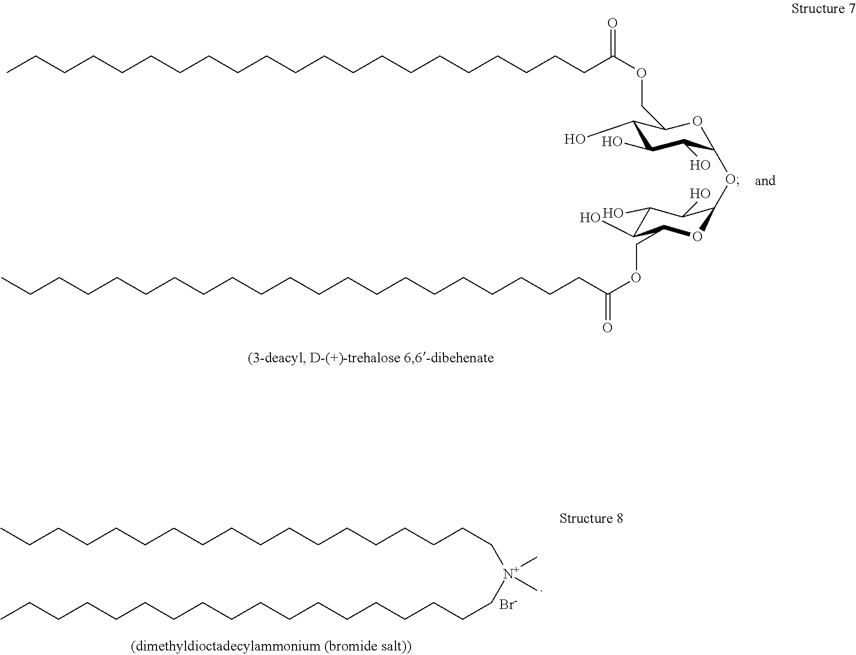

Additional examples of adjuvants and compositions thereof contemplated herein include those described in U.S. Pat. No. 9,241,988, which is incorporated by reference. In addition, bacterially-derived monophosphoryl lipid A, such as that provided by Avanti Polar Lipids, Inc. (Avanti No. 699200), may also be used.

In addition to naturally derived adjuvants, further examples of adjuvants contemplated for use herein include synthetically-derived adjuvants such as, for example, structural analogues of monophosphoryl lipid A including those available from Croda International Plc, including 3D(6-acyl)-PHAD™, 3D-PHAD™, and PHAD™.

Therapeutically effective amounts of adjuvants contemplated for use herein can be about 1 μg to about 100 μg, about 50 μg to about 90 μg, about 10 μg to about 80 μg, about 15 μg to about 70 μg, or about 20 μg to about 50 μg per dose, or any other amount that treats or assists in the treatment of a subject receiving the adjuvant. For example, a therapeutically effective amount of an adjuvant can be about 20 μg. For example, therapeutically effective amounts of a TLR4 agonist can be about 0.01 to about 20 mg/kg, or about 0.1 to about 10 mg/kg, or about 0.5 to about 5 mg/kg.

In some embodiments, the radiotherapy contemplated for use herein can be external beam radiation therapy (EBRT) and/or internal radiation therapy such as brachytherapy and radiopharmaceuticals. In some preferred embodiments, the radiotherapy is EBRT.

In some embodiments, therapeutically effective amounts of radiotherapy can range from about 2 to about 20 Gy.

In some embodiments, the therapeutically effective amount of radiotherapy is administered in a gradient dose of about 1 Gy/min, about 2 Gy/min, or any other amount that treats or assists in the treatment of a subject receiving the adjuvant.

In some embodiments, the therapeutically effective amount of radiotherapy is administered via continuous decay of a radiopharmaceutical.

In some embodiments, the radiotherapy may be combined with or substituted for other locally ablative therapies such as radiofrequency ablation, microwave ablation, and/or cryoablation techniques.

In some embodiments, the immune checkpoint inhibitor includes one or more therapeutic agents that inhibit CTLA-4, PD-1, and/or PD-L1. Examples of therapeutic agents that are contemplated herein include antibodies and antigen-binding fragments thereof, as well as small molecule inhibitors. For example, an immune checkpoint inhibitor contemplated for use herein can be an anti-CTLA-4 antibody. Specific anti-CTLA-4 antibodies contemplated for use herein are known in the art.

Therapeutically effective amounts of immune checkpoint inhibitors contemplated for use herein can be about 1 μg to about 400 μg, about 20 μg to about 300 μg, about 40 μg to about 200 μg, about 15 μg to about 70 μg, or about 20 μg to about 50 μg per dose, or any other amount that treats or assists in the treatment of a subject receiving the adjuvant. For example, a therapeutically effective amount of an immune checkpoint inhibitor can be about 200 μg. For example, therapeutically effective amounts of immune checkpoint inhibitors can be about 1.0 to about 50 mg/kg, or about 5 to about 25 mg/kg, or about 10 to about 20 mg/kg, or about 5 mg/kg, or about 10 mg/kg, or about 15 mg/kg, or about 20 mg/kg.

In another embodiment, the present disclosure provides a method of treating cancer in a subject in need thereof. The method includes the steps of administering a therapeutically effective amount of a TLR4 agonist to a tumor of the subject, wherein the TLR4 agonist is administered intratumorally. The method further includes administering a therapeutically effective amount of EBRT to the tumor, administering a therapeutically effective amount of an anti-CTLA-4 antibody to the subject, and at least one of reducing tumor volume, increasing overall survival of the subject, and increasing complete response rate in the subject. Additional outcomes that are contemplated herein include increasing rates of local control, progression free survival, and decreasing rates of new distant metastases.

Any type of cancer that has a solid tumor can be treated according to the methods described herein. For example, cancers that can be treated include melanoma and prostate cancer, among others.

In another embodiment, the present disclosure provides methods of treating cancer in a subject in need thereof that increase the production of Th1-associated, IgG2c anti-tumor antibodies associated with solid tumors. In another embodiment, the present disclosure provides methods of treating cancer in a subject in need thereof that induce a systemic anti-tumor immune response.

In some embodiments, a method of the present disclosure can administer one or more adjuvants before, at the same time as, or after administration of one or more immune checkpoint inhibitors. Components of the compositions to be administered can be administered via the same route of administration (e.g., intravenously) or via different routes of administration (e.g., one component is administered intravenously and another component is administered intratumorally). Any variation of timing and/or route of administration is contemplated herein.

Compositions

In other embodiments, the present disclosure provides therapeutic and pharmaceutical compositions (which can be referred generally to as “compositions”) for treating cancer. For example, a composition contemplated herein can include a therapeutically effective amount of an adjuvant, and/or a therapeutically effective amount of an immune checkpoint inhibitor, and a pharmaceutically acceptable carrier or diluent.

For example, compositions contemplated herein can include therapeutically effective amounts of one or more TLR4 agonists, such as monophosphoryl lipid A, monophosphoryl lipid A-504, monophosphoryl tri-acyl lipid A, monophosphoryl 3-deacyl lipid A, monophosphoryl tetra-acyl lipid A, monophosphoryl hexa-acyl lipid A, 3-deacyl, D-(+)-trehalose 6,6′-dibehenate, and dimethyldioctadecylammonium (bromide salt).

Further, compositions contemplated herein can include therapeutically effective amounts of one or more immune checkpoint inhibitors, such as antibodies and antigen-binding fragments thereof or small molecules that inhibit CTLA-4, PD-1, and/or PD-L1.

A particular composition contemplated herein includes monophosphoryl lipid A and a pharmaceutically acceptable carrier or diluent. The composition can also include an anti-CTLA-4 antibody.

Compositions as described herein can be formulated as separate compositions that are given simultaneously or sequentially, or combined into a single composition just prior to administration to a subject. Alternatively, compositions contemplated here can refer to a single composition that includes all components. In certain embodiments, compositions of the present disclosure can include one or more secondary therapeutic agents. Examples of suitable secondary therapeutic agents include intratumorally injected nanoparticles, microparticles, oncolytic viruses, immunotherapies such as cytokines, monoclonal antibodies, and immunocytokines, and chemotherapies such as, for example, cyclophosphamide.

Compositions of the present disclosure can be formulated for virtually any mode of administration, including, for example, injection, intratumoral administration, transdermal, oral, topical, ocular, buccal, systemic, nasal, rectal, vaginal, etc., or a form suitable for administration by inhalation or insufflation. Compositions that can be delivered (e.g., are formulated to be administered) intravenously, intratumorally, intraperitoneally, and/or intratracheally are also contemplated herein.

In some embodiments, a composition of the present disclosure is included in a pharmaceutical composition having at least one pharmaceutically acceptable carrier, solvent, adjuvant, or diluent.

The term “pharmaceutical composition” can be used in its widest sense, encompassing all pharmaceutically applicable compositions containing at least one active substance, and optional carriers, adjuvants, constituents, etc. The term “pharmaceutical composition” also encompasses a composition comprising an active substance in the form of a derivative or pro-drug, such as a pharmaceutically acceptable salt and/or ester. The manufacture of pharmaceutical compositions for different routes of administration falls within the capabilities of a person skilled in medicinal chemistry. The exact nature of the carrier, excipient, or diluent used in a pharmaceutical composition will depend upon the desired use for the pharmaceutical composition. The pharmaceutical composition can optionally include one or more additional compounds, such as therapeutic agents or other compounds, as described herein elsewhere.

The compositions described herein may be administered orally, topically, parenterally, by inhalation or spray, or rectally in dosage unit formulations containing conventional non-toxic pharmaceutically acceptable carriers, adjuvants, and vehicles. The term parenteral as used herein includes percutaneous, subcutaneous, intratumoral, intravascular (e.g., intravenous), intramuscular, or intrathecal injection or infusion techniques and the like.

Formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The therapeutic compositions described herein, or pharmaceutical compositions thereof, will generally be used in an amount effective to achieve the intended result, for example in an amount effective to treat or prevent the particular disease being treated (e.g., a therapeutically effective amount). By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally can include halting or slowing the progression of the disease.

The amount of therapeutic composition administered can be based upon a variety of factors, including, for example, the particular condition being treated, the mode of administration, whether the desired benefit is prophylactic and/or therapeutic, the severity of the condition being treated and the age and weight of the patient, the genetic profile of the patient, and/or the bioavailability of the particular therapeutic composition, etc.

Determination of an effective dosage of compound(s) for a particular use and mode of administration is well within the capabilities of those skilled in the art. Effective dosages can be estimated initially, for example, from in vitro activity and metabolism assays. For example, an initial dosage of a therapeutic composition for use in animals can be formulated to achieve a circulating blood or serum concentration of the therapeutic composition that is at or above an EC₅₀ of the particular therapeutic composition as measured in an in vitro assay. Calculating dosages to achieve such circulating blood or serum concentrations taking into account the bioavailability of the particular therapeutic composition via the desired route of administration is well within the capabilities of skilled artisans. Initial dosages of therapeutic composition can also be estimated from in vivo data, such as animal models. Animal models useful for testing the efficacy of the therapeutic composition to treat or prevent the various diseases described above are well-known in the art. Animal models suitable for testing the bioavailability of the therapeutic composition are also well-known. Ordinarily skilled artisans can routinely adapt such information to determine dosages of particular therapeutic compositions suitable for human administration.

In addition to amounts of active agents described herein elsewhere (e.g., adjuvants and ICI agents or chemotherapies), dosage amounts can also be in the range of from about 0.0001 mg/kg/day, 0.001 mg/kg/day, or 0.01 mg/kg/day to about 100 mg/kg/day, but may be higher or lower, depending upon, among other factors, the activity of the therapeutic agent, the bioavailability of the therapeutic composition, other pharmacokinetic properties, the mode of administration and various other factors, including particular diseases being treated, the site of the disease within the body, the severity of the disease, the genetic profile, age, health, sex, diet, and/or weight of the subject. Dosage amount and interval can be adjusted individually to provide levels of the therapeutic composition which are sufficient to maintain a desired therapeutic effect. For example, a therapeutic composition can be administered once per week, several times per week (e.g., every other day), once per day or multiple times per day, depending upon, among other things, the mode of administration, the specific indication being treated and the judgment of the prescribing physician. In cases of local administration or selective uptake, such as intratumoral injection, the effective local concentration of therapeutic compositions may not be related to plasma concentration. Skilled artisans will be able to optimize effective dosages without undue experimentation.

In a further aspect, contemplated herein are kits including one or more compositions to be administered according to the methods of treatment described herein. Such contemplated kits can further include instructions indicating how to administer the one or more compositions to be applied.

### Examples

The Examples that follow are illustrative of specific embodiments of the disclosure, and various uses thereof. They are set forth for explanatory purposes only and should not be construed as limiting the scope of the disclosure in any way.

### Example 1: Local TLR4 Stimulation Augments In Situ Vaccination Through Induction of CD8 T-Cell Independent Th1 Polarization

### Overview

Background: Radiation therapy (RT) has been demonstrated to generate an in situ vaccination (ISV) effect in murine models and in cancer patients; however, this has not routinely translated into enhanced clinical response to immune checkpoint inhibition (ICI). We investigated whether the commonly used vaccine adjuvant, monophosphoryl lipid A (MPL) could augment the ISV regimen consisting of combination RT and ICI.

Materials/Methods: We used syngeneic murine models of melanoma (B78) and prostate cancer (Myc-CaP). Tumor bearing mice received either RT (12 Gy, day 1), RT+anti-CTLA4 (C4, day 3, 6, 9), MPL (20 μg IT injection days 5, 7, 9), RT+C4+MPL, or PBS control. To evaluate the effect of MPL on the irradiated tumor microenvironment, primary tumor with tumor draining lymph nodes were harvested for immune cell infiltration analysis and cytokine profiling, and serum was collected for analysis of anti-tumor antibody populations.

Results: Combination RT+C4+MPL significantly reduced tumor growth, increased survival and complete response rate compared to RT+C4 in both B78 and Myc-CaP models. MPL favorably reprogrammed the irradiated tumor-immune microenvironment towards M1 macrophage and Th1 TBET+CD4+ T cell polarization. Furthermore, MPL significantly increased intratumoral expression of several Th1 and M1 associated proinflammatory cytokines. In co-culture models, MPL-stimulated macrophages directly activated CD8 T cells and polarized CD4 cells towards the Th1 phenotype. MPL treatment significantly increased production of Th1-associated, IgG2c antitumor antibodies which were required for and predictive of anti-tumor response to RT+C4+MPL and enabled macrophage-mediated antibody-dependent direct tumor cell killing by MPL-stimulated macrophages. Macrophage-mediated tumor cell killing was dependent on FcγR expression. In metastatic models, RT and MPL generated a systemic anti-tumor immune response that augmented response to ICIs. This was dependent on macrophages and CD4+ but not CD8+ T cells.

Conclusions: We report the potential for MPL to augment the ISV effect of combination RT+C4 through FcγR, macrophage, and TBET+CD4+Th1 cell dependent mechanisms. To our knowledge this is the first report describing generation of a CD8+ T cell-independent, Th1 polarized, systemic anti-tumor immune response with subsequent generation of immunologic memory. These findings support the potential for vaccine adjuvants to enhance the efficacy of in situ tumor vaccine approaches.

### Introduction

The majority of patients with cancer will receive radiation therapy at some point during their clinical care (1). While previously thought of as primarily a cytotoxic therapy, growing evidence suggests that radiation has a variety of immunomodulatory effects within the tumor microenvironment. Radiation (RT) can induce immunogenic tumor cell death and release of tumor-specific antigens (2, 3), upregulation of immune susceptibility markers such as Fas and MHC-I (4, 5), and production of inflammatory cytokines such as type 1 interferon (6-8), as well as other inflammatory cytokines and damage-associated molecular patterns that influence immune cell trafficking and activation (9-11). Through these mechanisms RT can help generate an in situ vaccination effect, converting the patient's own tumor into a nidus of enhanced antigen presentation to generate a more diverse tumor-specific T cell response that can be propagated to distant, out of RT field sites of disease (i.e., an abscopal response) (12-16).

In contrast, radiation also induces changes within the tumor microenvironment that are potentially detrimental to the development of anti-tumor immunity. These can include blunting of effector immune cell infiltration within the tumor by recruiting suppressive regulatory T cells as well as increased infiltration and activation of inhibitory macrophage and myeloid-derived suppressor cell lineages (17-21). These inhibitory mechanisms in part likely underlie the clinical observation that abscopal responses following RT monotherapy are exceedingly rare (22). Targeting such detrimental effects is one approach whereby immunotherapies may be used to augment the efficacy of radiotherapy.

Immune checkpoint inhibitors (ICI; e.g., anti-PD-L1 and anti-CTLA-4 inhibitors), are a class of immunotherapies that modulate immune tolerance of a tumor by blocking specific inhibitory receptor-ligand interactions on the surface of immune cells and thereby overcoming T cell inhibition or exhaustion (23). In patients with highly immunogenic tumors such as some melanomas, ICIs can restore efficacy to the anti-tumor immune response, sometimes resulting in complete and durable tumor regression even in settings of advanced metastatic disease (24-28). However, ICIs have not shown clinical benefit in the treatment of poorly immunogenic tumors such as prostate cancer that are characterized by low levels of T cell infiltrate and low mutation burden resulting in few mutation-created neo-antigens (29-32). Additionally, even patients with highly immunogenic tumors that initially respond to ICIs often exhibit disease progression over time (33).

Several groups have taken advantage of the immunostimulatory effects of RT to improve response to ICI therapy with remarkable success in preclinical models (4, 34-40). In addition, through mechanistic preclinical studies it is becoming increasingly clear that to generate clinically meaningful abscopal responses with RT, combination with immunotherapies such as ICI will likely be required (7, 40-42). Early clinical studies combining RT with ICI have shown promise; however, clinical responses remain limited (16, 36, 43-45). Therefore, there is immediate clinical need to boost both the rates and depth of response to combination RT and ICI therapy.

Monophosphoryl lipid A (MPL) is a derivative of the lipopolysaccharide (LPS) component of the cell-wall of Salmonella entericais. MPL promotes immune activation in mice and humans through activation of toll-like receptor 4 (TLR4), with markedly reduced toxicity compared to LPS (46). Clinically, MPL is used as an adjuvant in several infectious disease vaccines including the HBV vaccine Fendrix (46) and the HPV vaccine Cervarix (47). Much like conventional vaccines, in situ vaccination regimens rely on promoting antigen recognition which may be enhanced through co-administration of adjuvants. Additionally, MPL may overcome further detrimental effects of RT that are not addressed with ICIs such as preventing the activation of inhibitory macrophage and myeloid-derived suppressor cell lineages. By promoting reprogramming of innate cell populations within the tumor microenvironment towards proinflammatory phenotypes, MPL may augment the anti-tumor response generated via combination RT and ICI and function as an adjuvant to in situ vaccination.

Here, the potential of MPL to function as an adjuvant to the in situ vaccine regimen of combination RT and anti-CTLA-4 in immunologically cold models of murine melanoma and prostate cancer was investigated. We demonstrate the capacity of intratumorally injected MPL to polarize CD4 T cells towards a Th1 phenotype, induce production of functional anti-tumor antibodies, and directly activate and polarize macrophages within the tumor microenvironment towards an M1 phenotype which enables macrophage mediated tumor cell direct killing through a Th1 CD4 T cell dependent mechanism, and promotes propagation of a systemic anti-tumor immune response independent of CD8 T cells.

### Materials and Methods

Study Design

The objectives of this work were to determine whether the conventional vaccine adjuvant MPL could enhance the anti-tumor response of the in situ vaccination regimen consisting of combination RT and anti-CTLA-4, as well as determine mechanisms whereby MPL enhances anti-tumor efficacy. For our studies, tumors were established intradermally in mice, external beam radiation was delivered, PBS or anti-CTLA-4 were injected intraperitoneally, PBS or MPL were injected intratumorally, and tumor growth and overall survival were recorded. Serum was collected and analyzed for the presence and characterization of anti-tumor antibodies, and the tumor and tumor draining lymph node were collected for immune infiltrate analysis. Mice were randomized to experimental groups/treatment the day before treatment initiation. Generally, experimental groups consisted of at least 5-6 mice, but in some experiments up to 10 were used. To determine the effects of MPL on immune cell populations, we harvested macrophages from bone marrow and isolated CD4 and CD8 cells from spleens for in vitro monoculture and co-culture in the presence of MPL. To test which immune cell populations were critical for anti-tumor efficacy, we used an antibody-mediated depletion of macrophages, NK cells, CD4, and CD8 T cells. To confirm the requirement for anti-tumor antibodies to generate a sufficient anti-tumor immune response, we used mice deficient in Fcγ receptor.

Cell Lines

The murine melanoma B78-D14 (B78) cell line, derived from B16 melanoma as previously described, was obtained from Ralph Reisfeld (Scripps Research Institute) in 2002 (63). The murine prostate cancer Myc-CaP cell line was obtained from ATCC. B78 and B16 cells were grown in RPMI-1640 and were supplemented with 10% FBS, 100 U/mL penicillin, and 100 μg/mL streptomycin. Myc-CaP cells were grown in DMEM and were supplemented with 10% FBS, 100 U/mL penicillin, and 100 μg/mL streptomycin. Cell line authentication was performed per ATCC guidelines using morphology, growth curves, and Mycoplasma testing within 6 months of use.

Murine Tumor Models

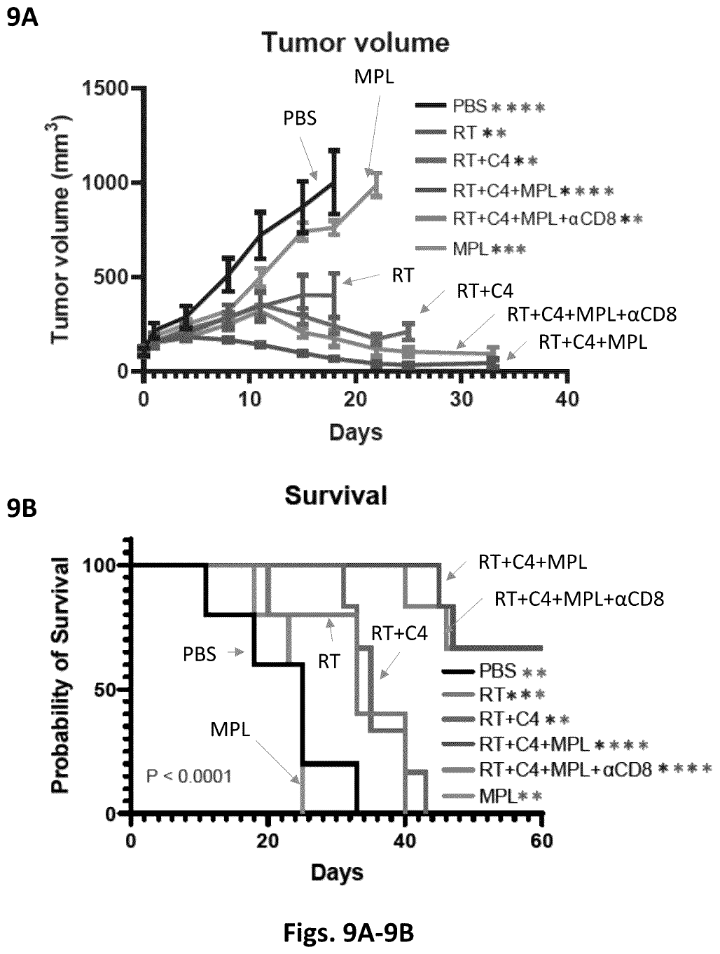

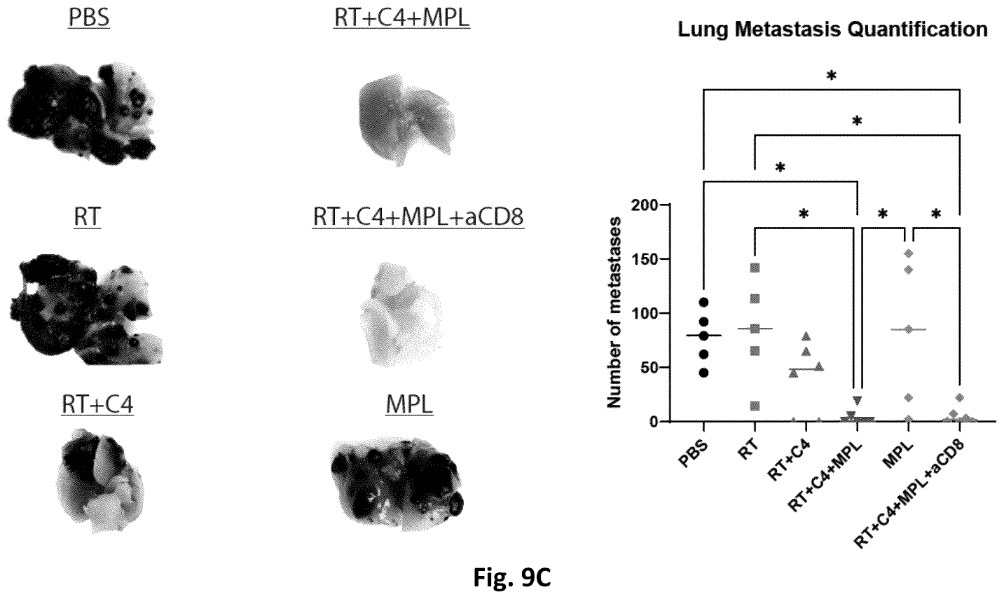

Mice were housed and treated under a protocol (protocol number M005670) approved by the Institutional Animal Care and Use Committee (IACUC) at the University of Wisconsin-Madison. Female C57BL/6, FcγR −/− (FcγR deficient C57BL/6.129P2-Fcer1gtm1Rav N12), and male FVBn mice were purchased at age 6 to 8 weeks from Taconic. Female TBET −/− (B6.129S6-Tbx21tm1Glm/J strain #:004648) were purchased from The Jackson Laboratory. B78 and Myc-CaP tumors were engrafted by subcutaneous flank injection of 2×106 and 1×106 tumor cells, respectively. Tumor size was determined using calipers and volume approximated as (width2×length)/2. Mice were randomized immediately before treatment when tumors were well-established (100-150 mm3), which occurred approximately 4 weeks after tumor implantation for B78 and 3 weeks for Myc-CaP. The day of radiation was defined as “day 1” of treatment. In the case of the metastatic model, 250,000 B16 cells were injected via tail vein injection immediately following radiation. Anti-CTLA-4 (IgG2c, clone 9D9, produced by NeoClone) was administered by 200 μg intraperitoneal injection on days 3, 6, and 9. MPL (Sigma Cat #SBR00012) was administered by 20 μg intratumoral injection on days 5, 7, and 9. T-cell, NK cell, and macrophage depletion was performed as previously described (12, 94). Depletion was confirmed on Day 15 of treatment (FIGS. 9A-9D). Mice were euthanized when tumor size exceeded 15 mm in longest dimension or whenever recommended by an independent animal health monitor for morbidity or moribund behavior.

Radiation

Delivery of RT in vitro was performed using a RS225 Cell Irradiator (Xstrahl). Delivery of RT in vivo was performed using an X-ray biological cabinet irradiator X-RAD 320 (Precision X-Ray, Inc). Mice were immobilized using custom lead jigs that exposed the right flank while shielding the rest of the mouse. In either case EBRT was prescribed to 12 Gy. The dose rate for RT delivery in all experiments was approximately 2 Gy/min. Dosimetric calibration and monthly quality assurance checks were performed on these irradiators by University of Wisconsin Medical Physics Staff.

Serum Antibody Analysis

To assess for the presence of anti-tumor antibodies in treated mice, blood was collected at days 15 and 30 for analysis as previously described (55). Briefly, serum components were isolated and frozen at −80° C. until ready for analysis, at which point serum was thawed and co-incubated with B78 cells for antibody labeling. Labeled cells were washed and tumor bound antibody was detected using secondary antibodies [anti-mouse IgG-FITC (405305; Biolegend), anti-mouse IgG1-PE (406607; Biolegend), anti-mouse IgG2b-PE (406708; Biolegend), anti-mouse IgG2c-FITC (NBP2-68518; Novus)] and a live dead vitality stain (DAPI).

Cell Culture

Macrophages were isolated from freshly harvested bone marrow as previously described (95-97). Briefly, isolated tibias were flushed with RMPI media and flowthrough was collected and centrifuged. The cell pellet was resuspended in RBC lysis buffer (Biolegend Cat #420302) and filtered (70 μM). Filtered cells were plated in non-tissue culture treated plates in Minimum Essential Medium Eagle-Alpha Modification (Alpha MEM) with Nucleosides supplemented with 10% FBS and 30 ng/mL M-CSF (Biolegend Cat #576408). After 24 hours, the culture supernatant containing macrophages was harvested and plated in tissue culture treated plates in Alpha MEM supplemented with 10% FBS and 120 ng/mL M-CSF.

CD4 and CD8 T cells were isolated from freshly harvested spleens of naïve mice. Spleens were homogenized, filtered (70 μM), and centrifuged. The cell pellet was resuspended in RBC lysis buffer and filtered (70 μM). CD4 and CD8 cells were sorted from total splenocytes using MACS column sorting (Miltenyi Biotec CD8a Cat #130-104-075, CD4 Cat #130-104-454) per manufacturer's instructions.

To determine direct effects of MPL on macrophages, CD4, and CD8 T cells, freshly isolated cells were cultured in 6-well plates containing Alpha MEM media (supplemented with 120 ng/mL M-CSF in the case of macrophages) in the presence of increasing amounts of MPL (5, 20, 100, and 500 ng/mL). After 24 hours cells were harvested, and RNA was isolated for analysis via qPCR.

In Vitro Co-Culture

Bone marrow derived macrophages were plated in 12 well plates (200,000 cells per well) containing Alpha MEM supplemented with 120 ng/mL M-CSF and treated with either 100 ng/mL MPL or PBS control. After 24 hours either CD4, CD8 or both were added (500,000 cells per well) to the culture. CD4 and CD8 cells were harvested 24 hours later and analyzed for activation markers using flow cytometry.

For co-culture with tumor cells, bone marrow derived macrophages (harvested from wild type or Fcγ receptor deficient C57BL/6 mice) were plated in 6 well plates (500,000 cells per well) containing Alpha MEM supplemented with 120 ng/mL M-CSF and treated with either 100 ng/ML MPL or PBS control. After 24 hours either B78 melanoma or Myc-CaP cells were added (200,000 cells per well). To test whether serum derived anti-tumor antibodies can activate macrophages, 5 μL of serum obtained from mice bearing B78 tumors rendered disease free was also added to the co-culture. After 24 hours cells were harvested and analyzed for polarization and activation markers using qPCR.

Cell Killing Assay

Bone marrow derived macrophages (harvested from wild-type or Fcγ receptor deficient C57BL/6 mice) were plated in 48 well plates (400,000 cells per well) containing Alpha MEM supplemented with 120 ng/mL M-CSF and treated with either 100 ng/ML MPL or PBS control. After 24 hours B78 melanoma cells were added (20,000 cells per well, 20:1 effector to target ratio) with or without 5 μL of serum obtained from mice bearing B78 tumors rendered disease free. After 24 hours cells were harvested via gentle scraping and washed with PBS twice. For cytotoxic CD4 T cell assessment, CD4 cells were isolated from spleens of B16 tumor bearing mice treated with either RT+C4+MPL or PBS control on day 15 following treatment and co-cultured with B16 cells that had received 0 or 12 Gy of RT. After 24 hours cells were harvested via gentle scraping and washed with PBS twice. In either case, a single cell suspension was labeled with CD45 antibody (anti-CD45-PE-Cy7, BioLegend, 157206) at 4° C. for 30 min and washed three times using flow buffer (2% FBS+2 mM EDTA in PBS). The single cell suspension was then labeled with the apoptotic marker Annexin V using the FITC Annexin V/Dead Cell Apoptosis Kit, (ThermoFisher Scientific Cat #V13242) per manufacturer's instructions. Flow cytometry was performed using an Attune NxT Flow Cytometer (ThermoFisher). Data were analyzed using FlowJo Software and percent of CD45− Annexin V+cells was quantified.

Gene Expression Analysis

Cells treated in vitro with MPL, RT, or the combination were washed with cold PBS, TRIzol™ reagent (ThermoFisher Scientific Cat #15596026) was added to the plate, and the cells were collected via scraping over ice. For analysis of tumor tissue, tumors were harvested, and samples were homogenized in TRIzol using a Bead Mill Homogenizer (Bead Ruptor Elite, Omni International Cat #19-040E). For in vitro and in vivo samples, total RNA was extracted using RNeasy Mini Kit (QIAGEN, Germany, Cat #74106) according to the manufacturer's instructions. Extracted RNA was subjected to complementary cDNA synthesis using QuantiTect Reverse Transcription Kit (QIAGEN, Germany, Cat #205314) according to the manufacturer's instructions. Quantitative polymerase chain reaction (qRT-PCR) was performed using Taqman Fast Advanced qPCR Master Mix. Thermal cycling conditions (Quantstudio 6, Applied Biosystems) included the UDG activation at 50° C. for 2 min, followed by Dual-Lock™ DNA polymerase activation stage at 95SC for 2 m followed by 40 cycles of each PCR step (denaturation) 95° C. for is and (annealing/extension) 60° C. for 20s. A melt curve analysis was done to ensure specificity of the corresponding qRT-PCR reactions. For data analysis, the Ct values were exported to an Excel file, and fold change normalized to untreated control samples was calculated using the ΔΔCt method. Hprt, was used as endogenous controls. A complete list of Taqman probes is included as Table 1.

TABLE 1
TaqMan Probes

| Gene Name | Gene Name Accession Number or Sequence |
|---|---|
| HPRT | Mm03024075_m1 |
| ARG1 | Mm00475988_m1 |
| NOS2 | Mm00440502_m1 |
| IL-1a | Mm00439620_ml |
| IL-1b | Mm00434228_m1 |
| IL-6 | Mm00446190_ml |
| IFNß1 | Mm00439552_s1 |
| IL-10 | Mm01288386_m1 |
| IFNγ | Mm01168134_m1 |
| TNFa | Mm00443258_m1 |
| CXCR3 | Mm00438259_m1 |
| CXCR4 | Mm01292123_m1 |
| Fcgr1 | Mm00438874_m1 |
| Fcgr2 | Mm00438875_ml |
| Fcgr3 | Mm01290524_m1 |
| Fcgr4 | Mm00519988_m1 |
| CD80 | F: TCTTTAGCATCTGCCGGGTG (SEQ ID NO: 1) |
| | R: GAGCCAATGGAGCTTAGGCA (SEQ ID NO: 2) |
| CD86 | F: CTTACGGAAGCACCCACGAT (SEQ ID NO: 3) |
| | R: CGTCTCCACGGAAACAGCAT (SEQ ID NO: 4) |
| MHC-II | F: GCCTAGTTATTGATGATCCAGGGT |
| | (SEQ ID NO: 5) |
| | R: AGAGACTTGAATTTGCCCTAACA |
| | (SEQ ID NO: 6) |

Flow Cytometry

Flow cytometry was performed as previously described (65), using fluorescent beads (UltraComp Beads eBeads, 176 Invitrogen, #01-2222-42) to determine compensation and fluorescence minus one (FMO) methodology to determine gating. For in vivo analysis, tumors and tumor draining lymph nodes were harvested and gently dissociated. For in vitro analysis, nonadherent CD4 and CD8 cells were collected from culture plates and washed with PBS twice. In either case total cells were treated CD16/32 antibody (BioLegend) to prevent non-specific binding. Live cell staining was performed using Ghost Red Dye 780 (Tonbo Biosciences) according to manufacturer's instruction. After live-dead staining, a single cell suspension was labeled with the surface antibodies at 4° C. for 60 min and washed three times using flow buffer (2% FBS+2 mM EDTA in PBS). For intracellular staining, the cells were fixed and stained for internal markers with permeabilization solution according to manufacturer's instructions (BD Cytofix/Cytoperm™). Flow cytometry was performed using an Attune NxT Flow Cytometer (ThermoFisher). Data was analyzed using FlowJo Software. Complete list of antibody targets, clones, and fluorophores is provided in Table 2.

TABLE 2
Flow Antibodies

| Name | Clone | Fluorophore | Cat no |
|---|---|---|---|
| CD8a | 53-6.7 | Alexa Fluor 700 | BioLegend 100730 |
| CD4 | RM4-5 | FITC | BioLegend 100510 |
| Ifnγ | XMG1.2 | PE | BioLegend 505808 |
| F4/80 | BM8 | PE/Dazzle 594 | BioLegend 123146 |
| CD80 | 16-10A1 | OE/Cyanine5 | BioLegend 104712 |
| CD25 | PC61 | Brilliant Violet 421 | BioLegend 102043 |
| I-A/I-E | M5/114.15.2 | Brilliant Violet 510 | BioLegend 107636 |
| (MHC-II) | | | |
| CD45 | 30-F11 | Brilliant Violet 605 | BioLegend 103140 |
| CD11b | M1/70 | Brilliant Violet 711 | BioLegend 101242 |
| CD206 | C068C2 | APC | BioLegend 141708 |
| CD11c | N418 | PE/Dazzle 594 | BioLegend 117348C |
| CD103 | 2E7 | PE/Cyanine7 | BioLegend 121426 |
| T-bet | 4B10 | Brilliant Violet 421 | BioLegend 644815 |
| PD-1 | RMP1-30 | APC | BioLegend 109112 |
| CD69 | H1.2F3 | Brilliant Violet 711 | BioLegend 104537 |
| FOXP3 | FJK-16s | PerCP-Cyanine5.5 | ThermoFisher |
| | | | 45-5773-82 |
| Gata-3 | TWAJ | PE | ThermoFisher |
| | | | 12-9966-42 |
| CXCR3 | CXCR3-173 | Brilliant Violet 510 | BioLegend 126528 |
| CXCR4 | L276F12 | PE/Dazzle 594 | BioLegend 146514 |
| IgG | Poly4053 | FITC | BioLegend 405305 |
| IgG1 | RMG1-1 | PE | BioLegend 406608 |
| IgG2c | Poly | FITC | Novus Biologicals |
| | | | NBP2-68518 |

Tumor Cytokine Multiplex Immunoassay

At day 15, tumors were harvested and weighed. Tumor samples (5 μL/mg) were lysed in 20% Cell Lysis Buffer with PMSF (Cell Signaling Technology) and supplemented with Halt Protease and Phosphatase Inhibitor Cocktail (Thermo Scientific). Each tumor was homogenized in bead beater tubes, and the lysate was stored at −80° C. The concentration of 32 cytokines and chemokines in the tumor lysates (MILLIPLEX MAP Mouse Cytokine/Chemokine Magnetic Bead Panel, Millipore) were determined by a multiplex immunoassay following manufacturer's instructions. The MAGPIX System (Millipore) was used to read the multiplex plate. Concentrations were determined using a standard curve and their respective median fluorescence intensity (MFI) readings (Milliplex Analyst, Millipore). The data underwent log and Z646 (Z) transformation followed by unbiased hierarchical clustering.

Statistical Analysis

Prism 8 (GraphPad Software) and R version 4.0.2 (The R Foundation) were used for all statistical analyses. One-way ANOVA with Tukey's honestly significant difference (HSD) test to adjust for multiple comparisons was used to assess statistical significance of observed mean differences in gene expression and immune cell quantification. For comparisons between two groups a Student's t-test was performed. For tumor growth analysis, a linear mixed model after log transformation of tumor volume was fitted on treatment and day. Day and the interaction between treatment and day were fixed effects. When testing differences in slopes of log transformed tumor volume, a Tukey adjustment for multiplicity was used. The Kaplan-Meier method was used to estimate the survival distribution for the overall survival. A Cox regression model was fitted, and pairwise comparison of the overall survival was made using a log-rank test with Benjamini-Hochberg adjustment of p-values between levels of factors. Chi-squared test was used to compare complete response rate.

Results

Intratumoral MPL Enhances Anti-Tumor Response Generated by Combination RT and Anti-CTLA-4

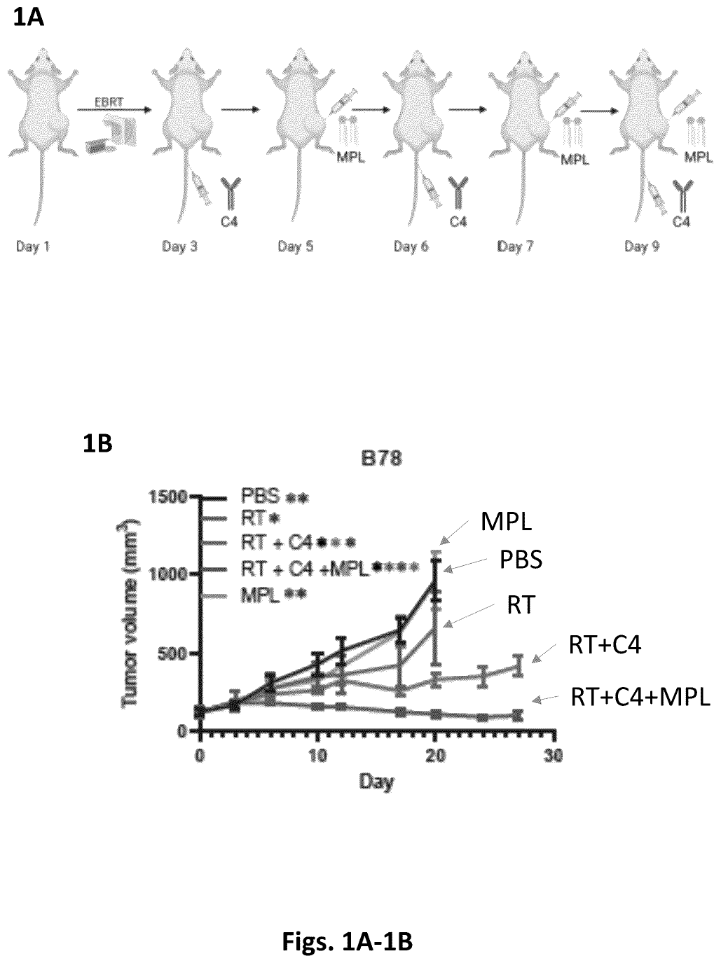

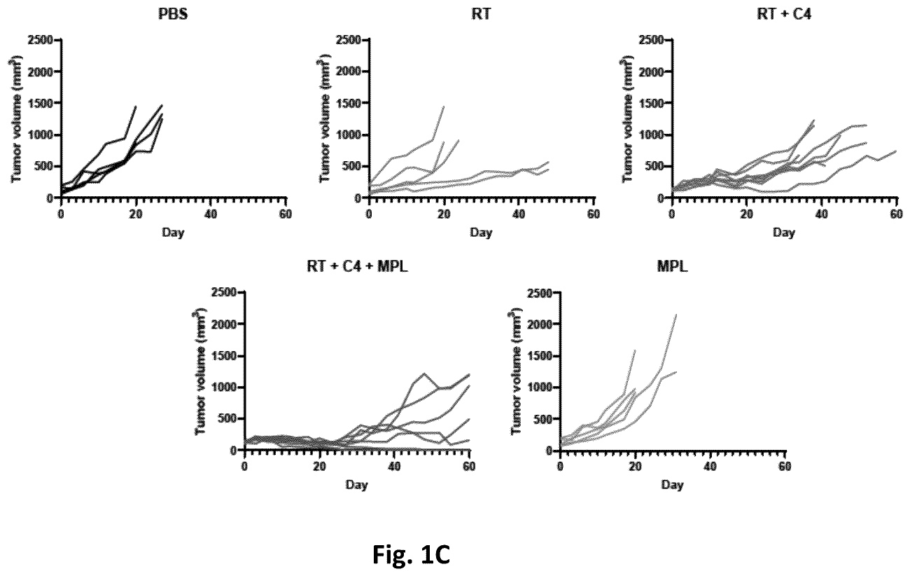

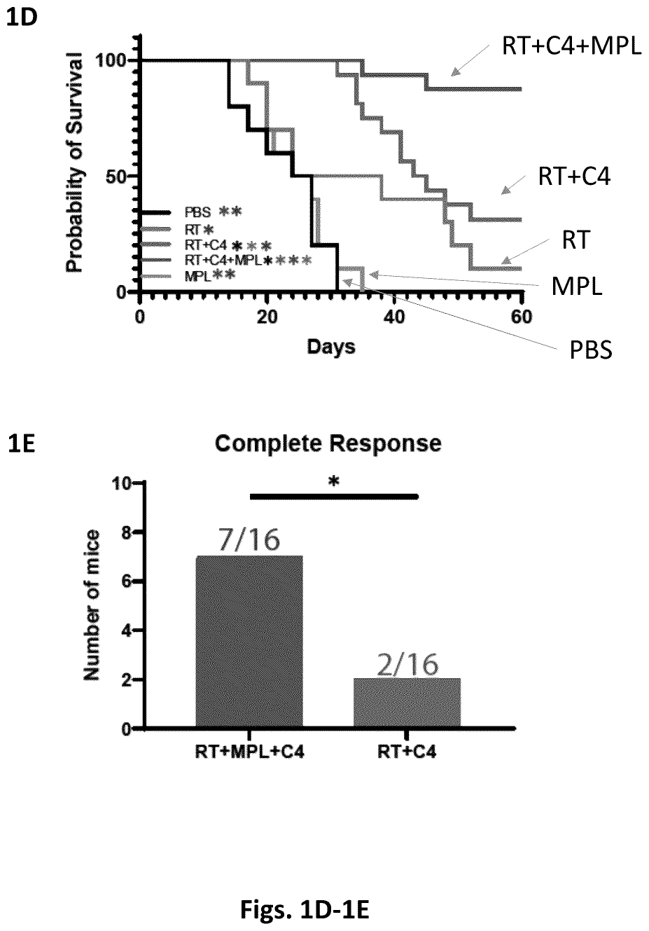

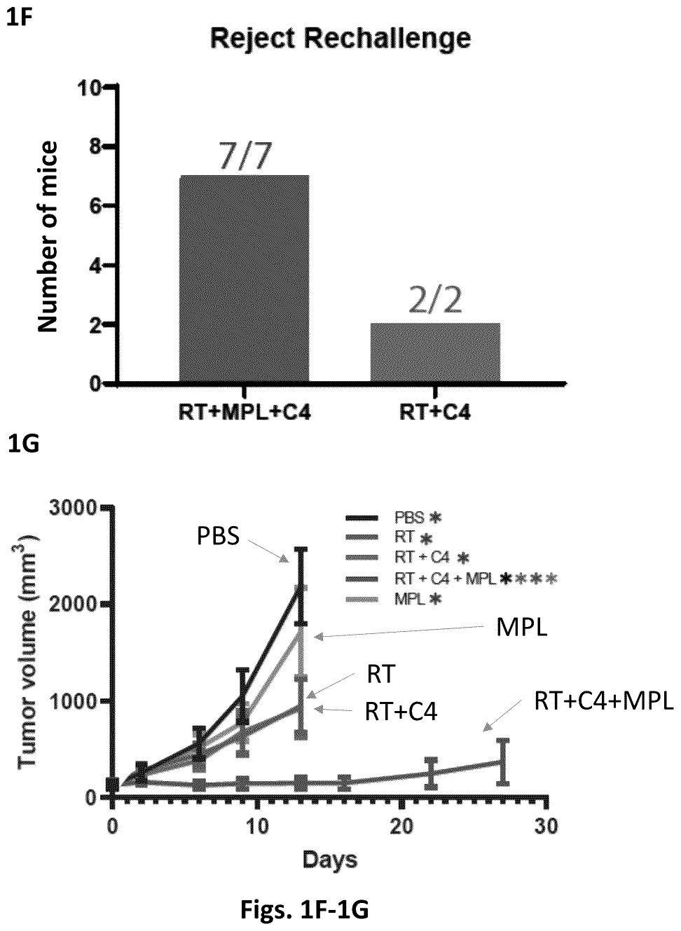

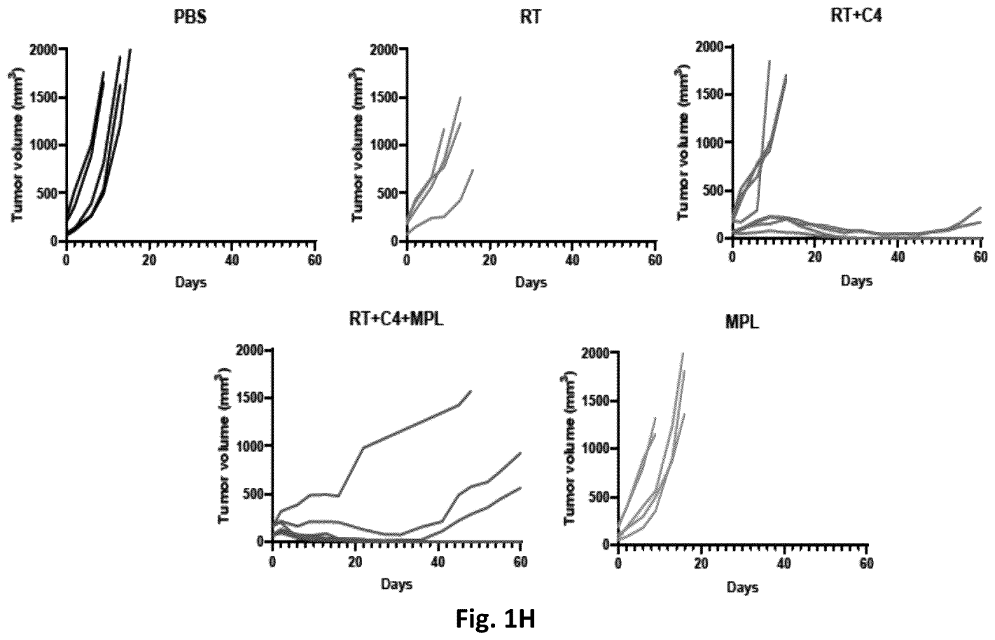

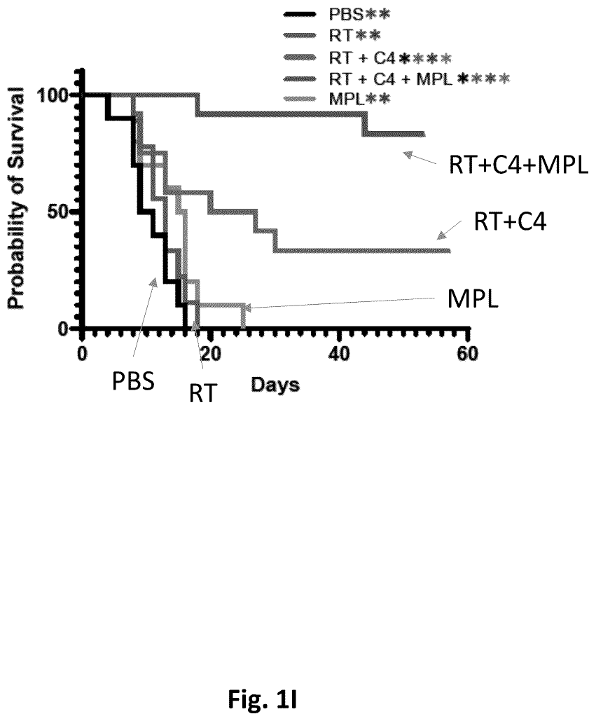

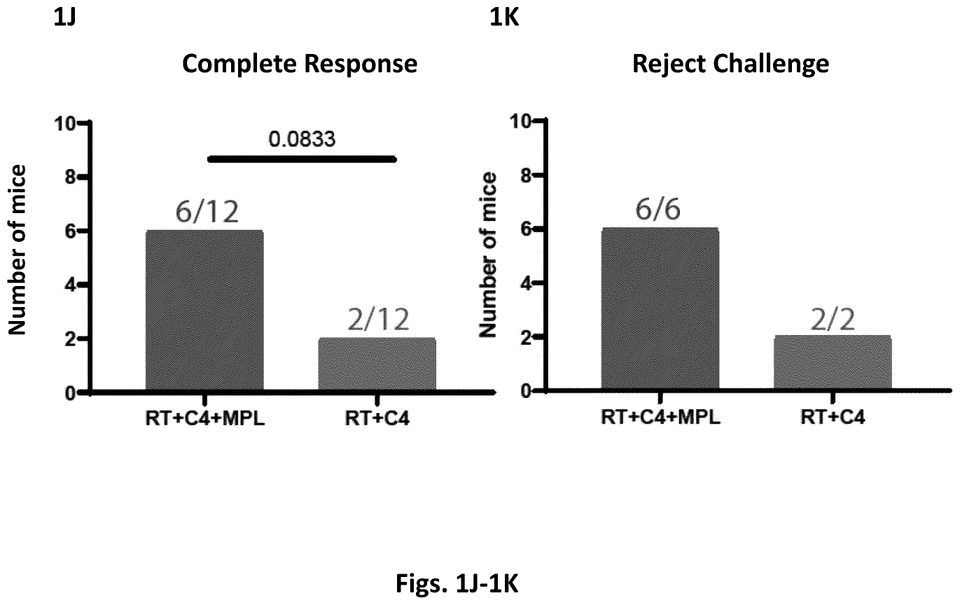

Clinically, melanoma is typified by high tumor mutation burden and consequently may be rich in mutation-created neo-antigens, often resulting in an endogenous anti-tumor immune response that is exhausted or otherwise rendered ineffective in clinically detected tumors (48). While some patients with melanoma respond well to ICIs, an absence of inflammatory signals, presence of suppressive immune lineages, limited tumor cell immune susceptibility (e.g. down regulation of MHC-I), and ineffective antigen presentation may result in functionally “cold” tumors that do not respond to ICIs alone (49-51). Therefore, to improve outcomes in patients resistant to ICI, we utilized the syngeneic B78 murine melanoma model. This model exhibits many hallmarks of resistance to ICI including low MHC-1 expression with low numbers of baseline tumor infiltrating lymphocytes (12, 52). In mice bearing B78 flank tumors, we tested the capacity of MPL to function as an adjuvant to combination therapy consisting of RT and anti-CTLA-4. We randomized mice to receive either RT (12 Gy delivered on day 1), RT+anti-CTLA-4 (C4; 200 μg intraperitoneal injection on days 3, 6, 9), RT+C4+MPL (20 μg intratumoral injection on days 5, 7, 9), RT+MPL, MPL+C4, C4 alone, MPL alone, or PBS control (FIG. 1A). The MPL dosing regimen was chosen to coincide with pre-peak, peak, and post-peak immune activation using type 1 interferon induction as a marker following RT in our model with peak expression occurring 7 days following RT (21). We observed a significant reduction in tumor volume with combination RT+C4+MPL compared to single agent treatment groups (PBS vs RT+C4+MPL p<0.001; RT vs RT+C4+MPL p<0.001; MPL vs RT+C4+MPL p<0.001) (FIGS. 1B, 1C). This resulted in a significant increase in overall survival (PBS vs RT+C4+MPL median survival 31 days vs UND, p<0.001; RT vs RT+C4+MPL median survival 31 days vs UND, p <0.001; MPL vs RT+C4+MPL median survival 31 days vs UND, p<0.001) (FIG. 1D). Compared to RT+C4, combination RT+C4+MPL resulted in a significant reduction in tumor volume (p=0.003) and increase in overall survival (RT+C4 vs RT+MPL+C4 median survival 44 days vs UND, p=0.002) (FIGS. 1B-1D). Moreover, the addition of MPL significantly increased the complete response (CR) rate generated by combination RT+C4 (RT+C4 vs RT+MPL+C4 CR % 12.5% vs 44%) (FIG. 1E). In both RT+C4 and RT+C4+MPL treatment groups, mice that were rendered disease free rejected rechallenge with B78 cells, demonstrating development of a specific anti-tumor immune response (FIG. 1F).

We then sought to test our combination treatment strategy in a separate model of prostate cancer. In contrast to melanoma, prostate tumors commonly arise from driver mutations or oncogenic translocations and these tumors have a low mutation burden, limited anti-tumor immune response, and poor response to ICIs (31, 53) even when delivered in combination with RT (32). We confirmed these findings using the syngeneic Myc-CaP prostate cancer model. Combination RT+C4 failed to significantly reduce tumor growth compared to RT alone (p=0.094) or PBS control (p=1). In contrast, we observed a significant reduction in tumor volume (p<0.001) and increase in overall survival (RT+C4 vs RT+MPL+C4 median survival 23.5 days vs UND, p=0.017) with the addition of MPL to RT+C4 (FIGS. 1G-1I). Additionally, we observed an increase in CR rate (RT+C4 vs RT+MPL+C4 CR % 16.7% vs 50%) which trended towards significance (p=0.0833) (FIG. 1J). Mice rendered disease free at day 90 following treatment rejected rechallenge, confirming immunological memory (FIG. 1K).

MPL Polarizes Towards Th1 Phenotype and is Predictive of Response to In Situ Vaccination

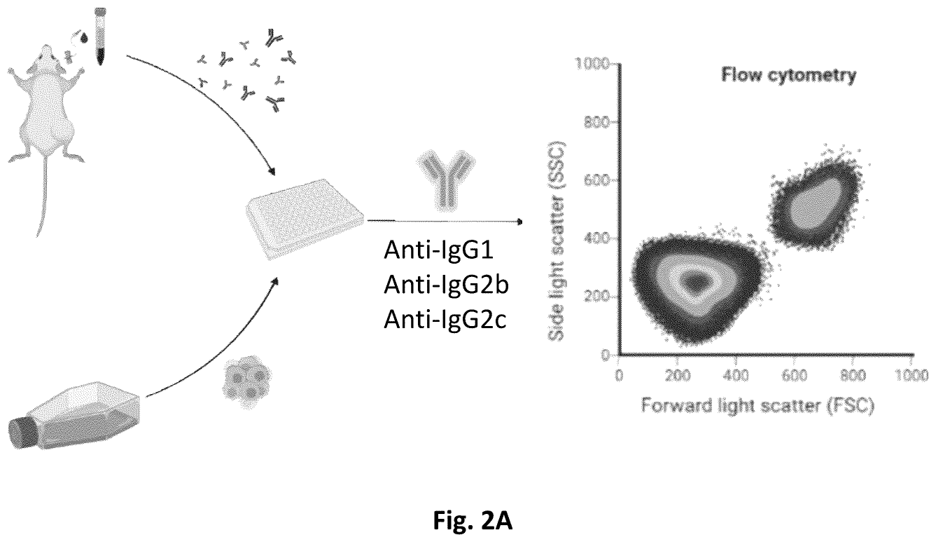

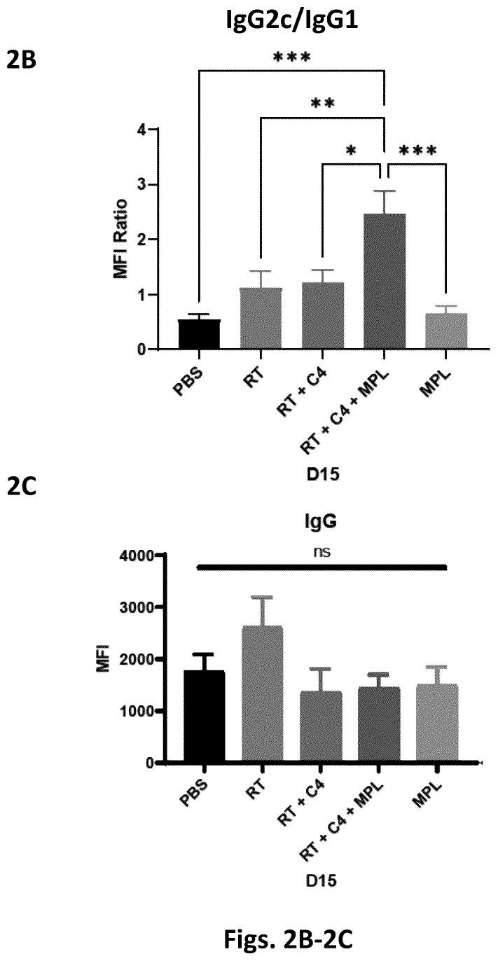

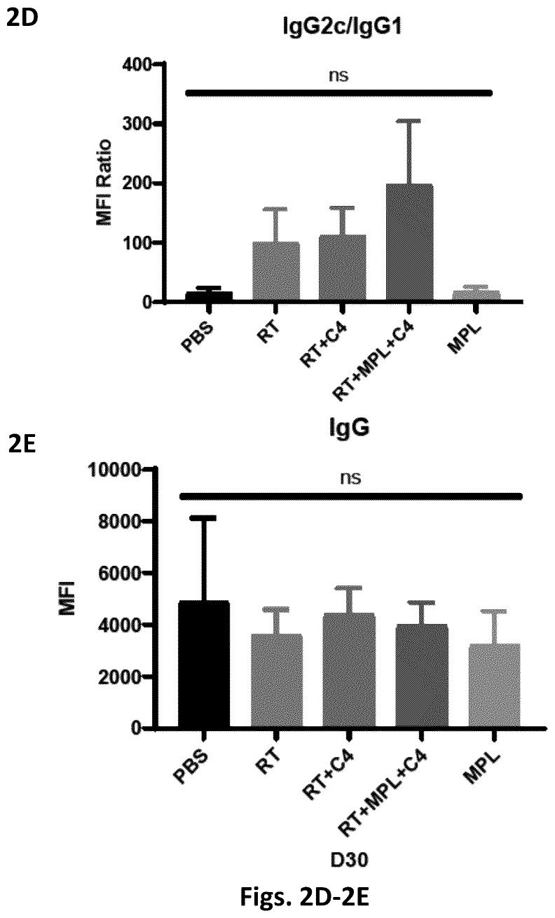

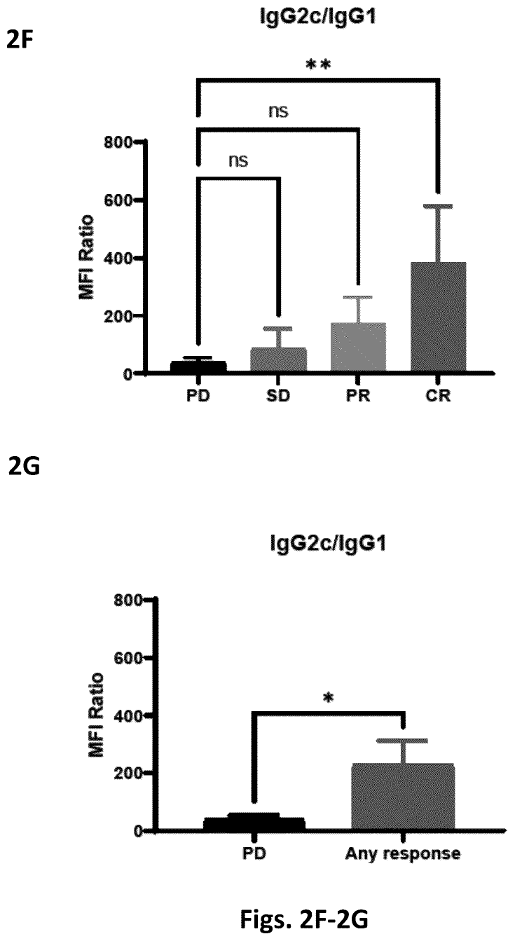

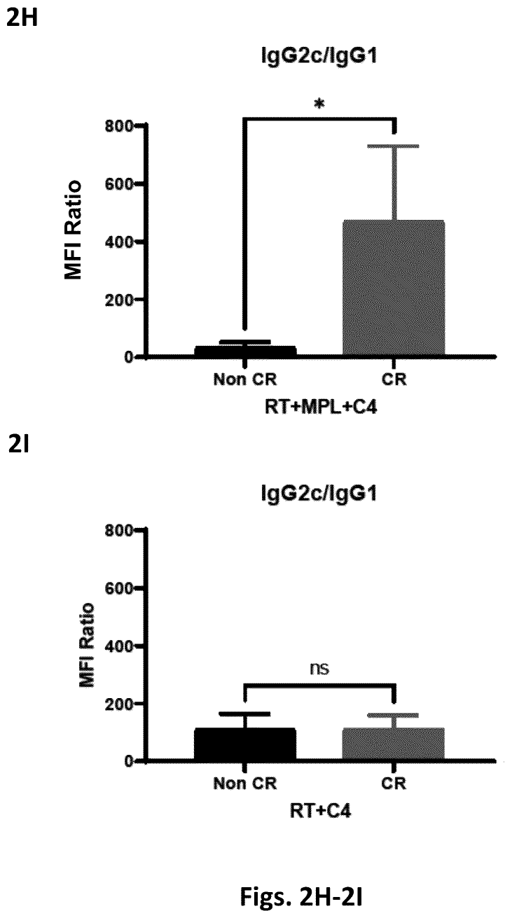

To test whether MPL polarizes CD4 cells to a Th1 phenotype in a radiated tumor microenvironment, we treated mice bearing B78 tumors with either PBS, RT, RT+C4, RT+C4+MPL, or MPL alone and collected serum at day 15 and 30 following RT. We chose day 15 and 30 as collection time points to coincide with previously observed peak intratumoral immune activation and production of anti-tumor antibodies, respectively, within our B78 tumor model (54, 55). We incubated B78 cells with isolated serum from these mice and detected anti-tumor antibody by flow cytometry. We then quantified levels of select IgG subclasses using fluorescently labeled secondary antibodies (anti-IgG, anti-IgG1, anti-IgG2c) via flow cytometry (FIG. 2A). In C57BL/6 mice, the antibody class IgG2c is associated with Th1 polarization whereas IgG1 is associated with Th2 polarization (56). Using IgG2c:IgG1 as a marker of Th1 polarization, we observed a statistically significant increase in the IgG2c:IgG1 ratio in the RT+C4+MPL group compared to all others at day 15 following treatment (FIG. 2B). We found that the total anti-B78 IgG antibody population was unchanged by treatment, when measured at day 15 (FIG. 2C). At day 30 the IgG2c/IgG1 ratio further increased by 100-fold compared to day 15, with RT+C4+MPL demonstrating the highest ratio compared to other groups which trended towards significance (FIG. 2D). We also found that the total anti-B78 IgG antibody population was unchanged by treatment, when measured at day 30 (FIG. 2E).

To determine whether the antibody class ratios correlated with depth of response, we classified the serum antibody populations by Response Evaluation Criteria in Solid Tumors (RECIST) criteria in a treatment agnostic manner. The IgG2c:IgG1 ratio was significantly increased in responding mice compared to mice with progressive disease, with the highest ratio in the CR group and second highest in the partial response (PR) group (FIG. 2F). In the RT 180+MPL+C4 group, mice with a CR had a significant increase in IgG2c/IgG1 compared to nonresponders (FIG. 2G), which likely underlies the loss of statistical significance comparing RT+MPL+C4 to other groups (FIG. 2D). This association of IgG2c:IgG1 ratio with CR was not seen in the RT+C4 group suggesting that the addition of MPL increases the complete response rate in a Th1 mediated fashion (FIG. 2H).

MPL Polarizes Macrophages Towards M1 Phenotype, and Promotes T Cell Activation

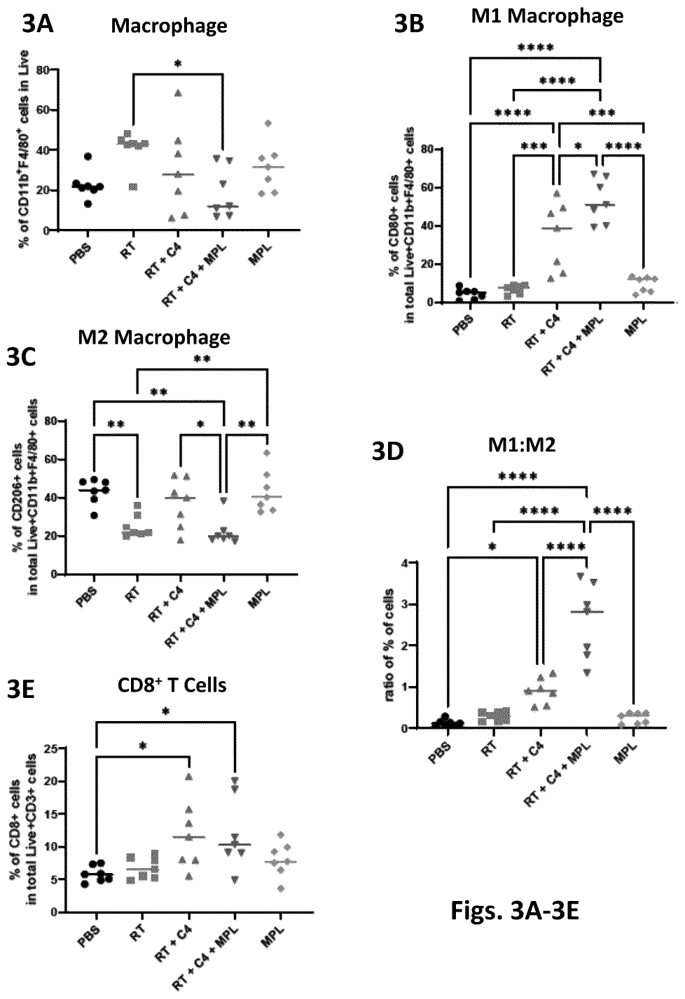

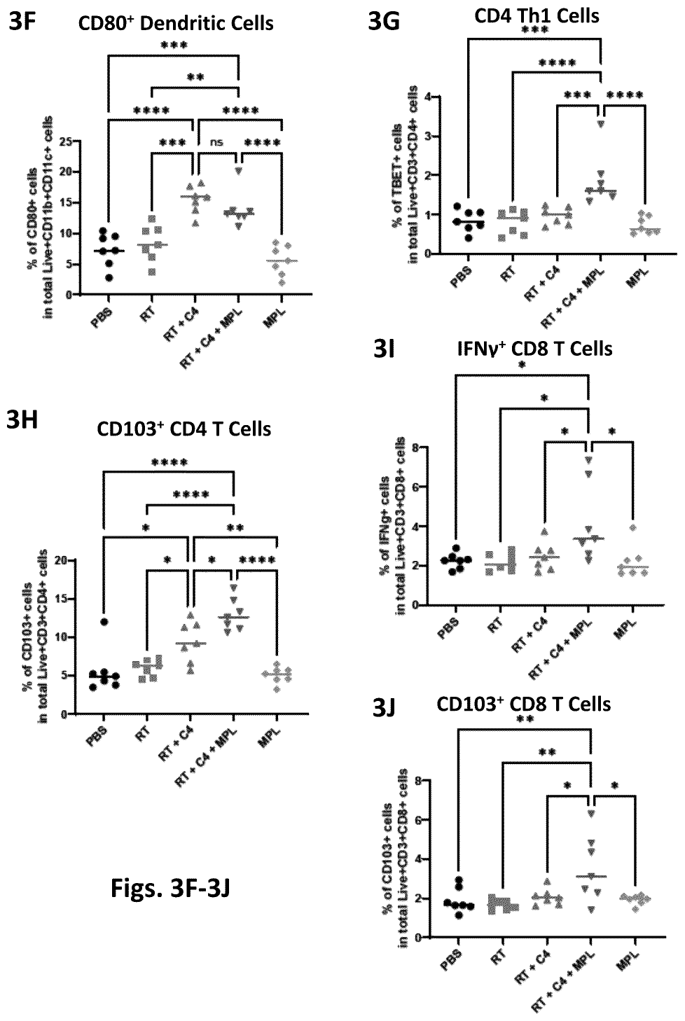

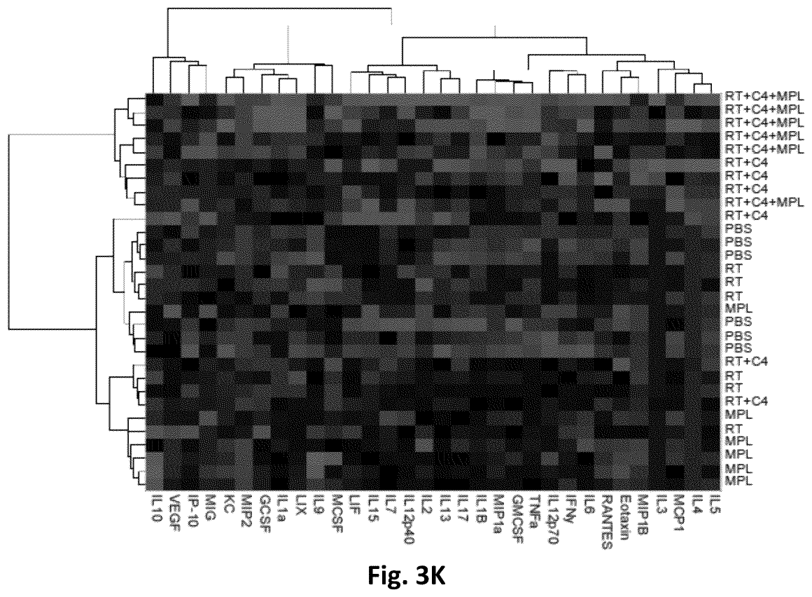

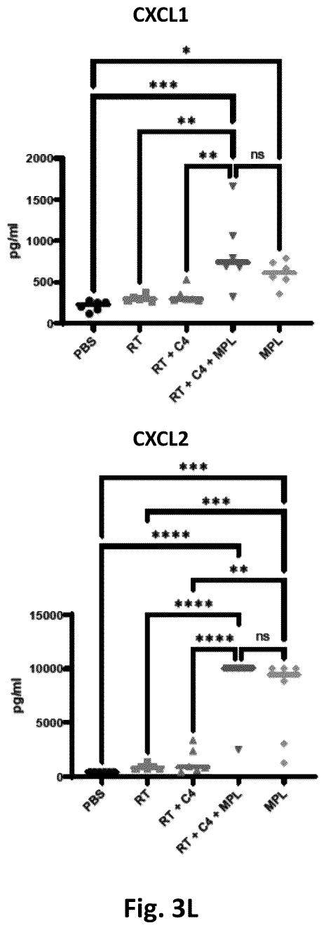

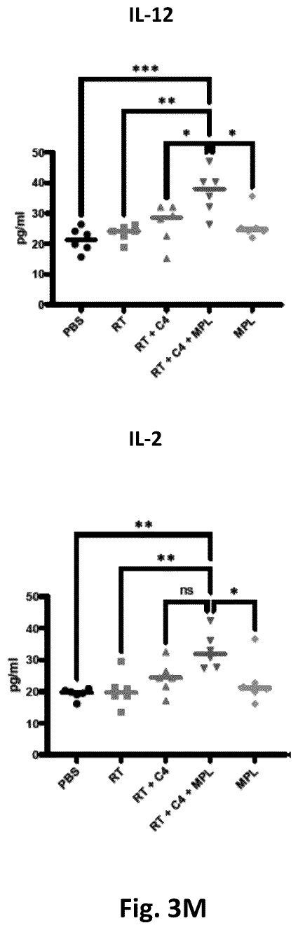

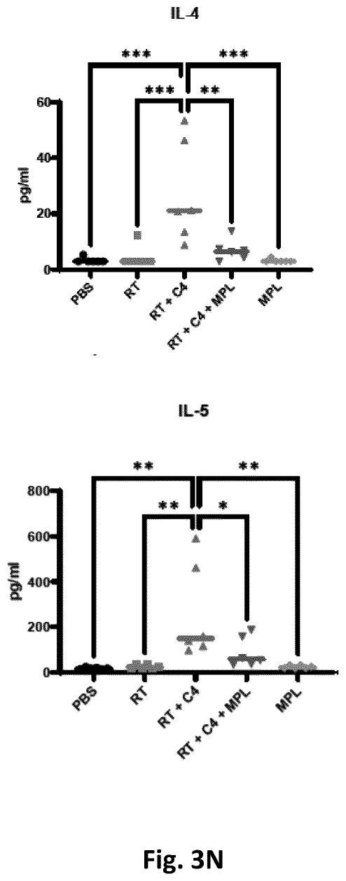

We next sought to determine the effects of MPL on the irradiated tumor immune microenvironment, as prior studies showed MPL predominantly activated M1 macrophages (57). We randomized B78 tumor bearing mice to either PBS, RT, RT+C4, RT+C4+MPL, or MPL alone and harvested tumors and the tumor draining lymph node on day 15 following RT. We observed a significant difference in number of macrophages in the RT+C4+MPL group compared to RT alone (FIG. 3A). Combination RT+C4 significantly increased the percentage of anti-tumor M1 macrophages (F4/80+CD11b+CD80+) out of total macrophages (CD11b+F480+) compared to PBS, RT, and MPL groups. This increase was further enhanced with the addition of MPL (FIG. 3B). Additionally, the percentage of M2 macrophages (F4/80+CD11b+CD206+) out of total macrophages (CD11b+F480+) was significantly decreased in the RT+C4+MPL group compared to PBS, RT+C4 and MPL groups (FIG. 3C). This resulted in a significant increase in the M1:M2 ratio in the RT+C4+MPL group compared to all others (FIG. 3D). Additionally, we observed a significant increase in number of CD8 T cells present in the tumor in both the RT+C4 and RT+C4+MPL groups compared to PBS control (FIG. 3E).

We then sought to determine whether MPL influenced antigen presentation in the context of the irradiated tumor immune microenvironment and to characterize CD4 Th populations in order to corroborate our anti-tumor antibody characterization findings following MPL treatment. Within the tumor draining lymph node we observed comparable increases in type 1 dendritic cells between RT+C4 and RT+C4+MPL, which were both significantly increased compared to PBS, RT, and MPL (FIG. 3F). The percentage of Th1 cells (CD4+TBET+) was significantly increased in the RT+C4+MPL group compared to all others, which is consistent with the increase in Th1-associated IgG2c antibody class switching we observe following RT+C4+MPL treatment (FIG. 3G). We observed significant increases in CD103+CD4+memory T cells in RT+C4 compared to PBS, RT, and MPL. This was further enhanced with the addition of MPL (FIG. 3H). Additionally, RT+C4+MPL generated significant increases in IFNγ+CD8 T cells and CD103+CD8+memory T cells compared to all other groups (FIGS. 3I, 3J).

We next profiled the cytokine repertoire within the irradiated tumor immune microenvironment following MPL treatment, focusing on TLR4 activation, Th polarization, and macrophage polarization given our tumor immune cell infiltration findings. We observed significant increases in keratinocytes-derived chemokine (KC, CXCL1) and Macrophage Inflammatory Protein 2 (MIP2, CXCL2) in both RT+C4+MPL and MPL groups (FIGS. 3K, 3L), consistent with TLR4 signaling activation (58). RT+C4+MPL significantly increased expression of Th1 associated cytokines IL-12 compared to all other groups and IL-2 compared to all other groups except RT+C4 which trended towards significance (FIG. 3M). RT+C4 favored Th2 signaling and led to significant increases in Th2 cytokines IL-4 and IL-5 compared to all other groups (FIG. 3N). Lastly, RT+C4+MPL significantly increased several pro-inflammatory cytokines associated with M1 polarization including I 224 L-1α, IL-10, Lipopolysaccharide-induced CXC Chemokine (LIX, CXCL5), TNFα, and GM-CSF compared to all other groups (FIG. 3O).

MPL Induces CD8 T Cell Activation Through Direct Stimulation and M1 Polarization of Macrophages

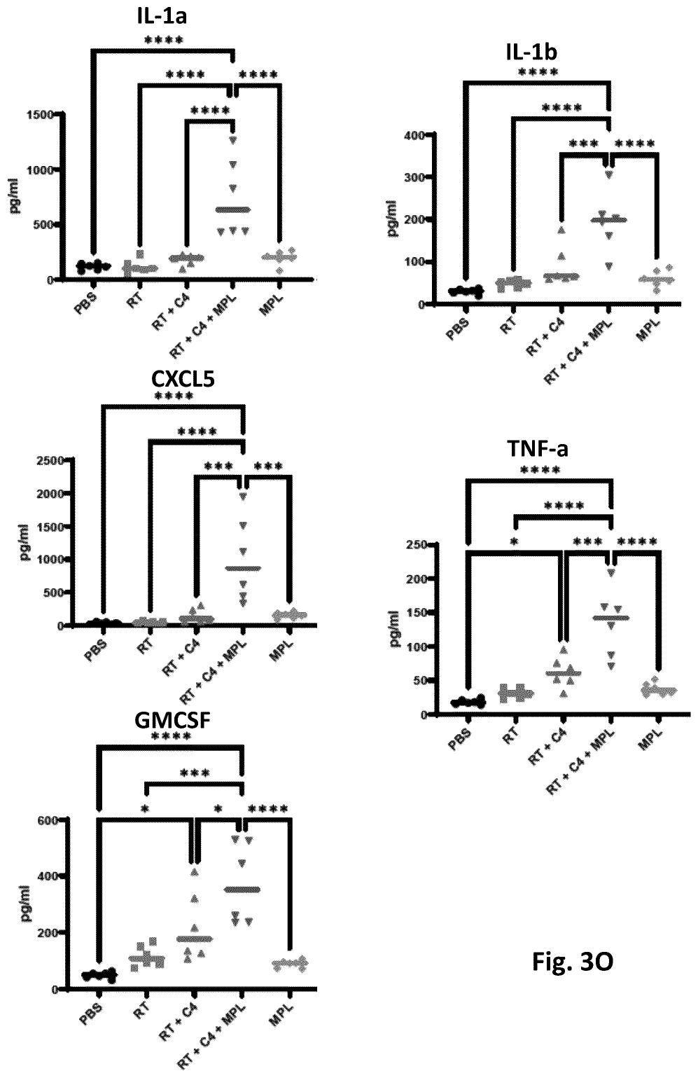

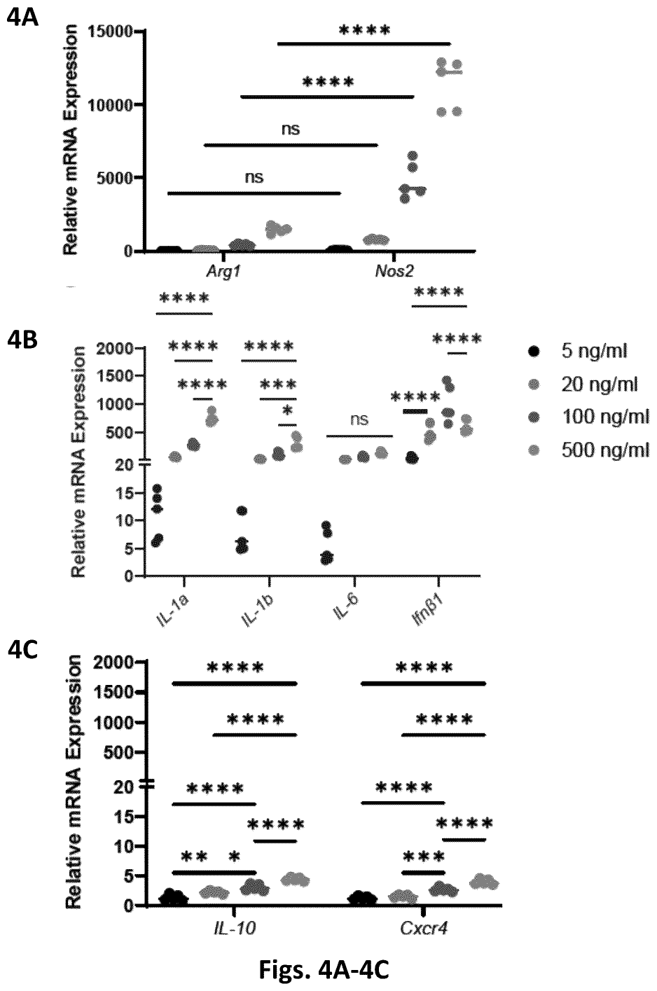

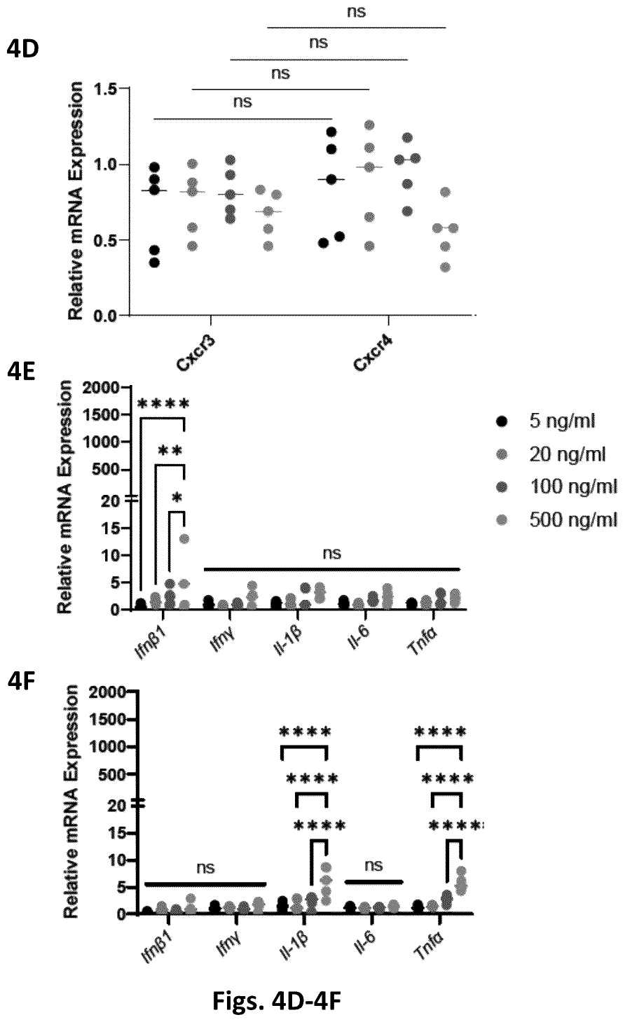

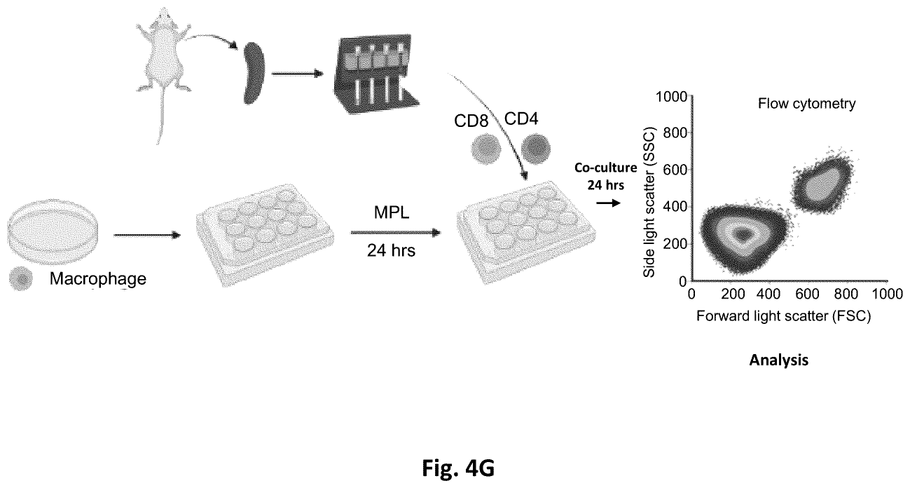

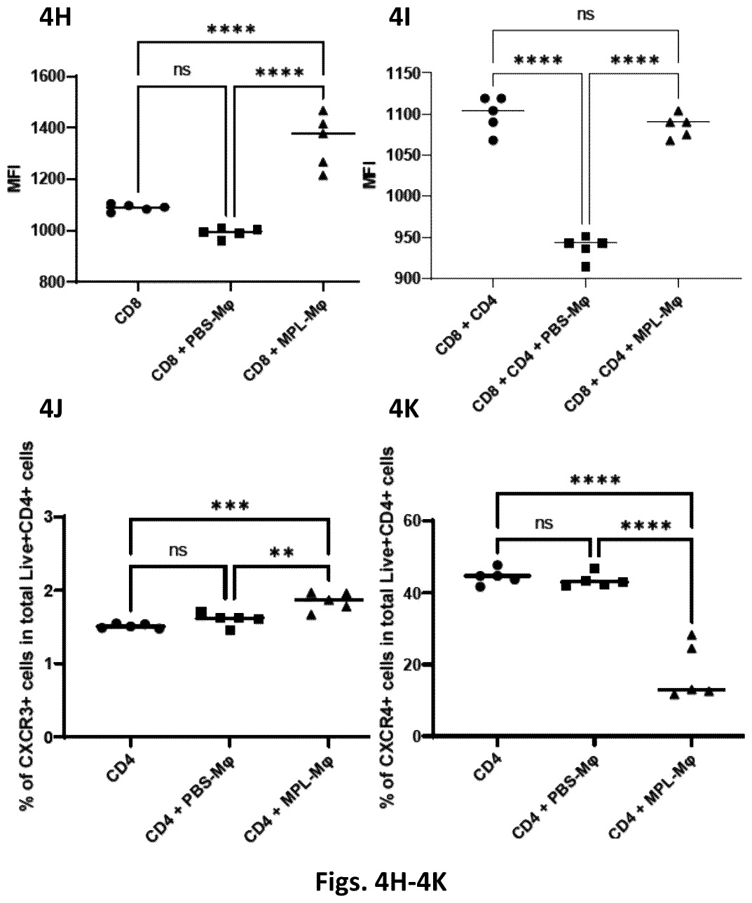

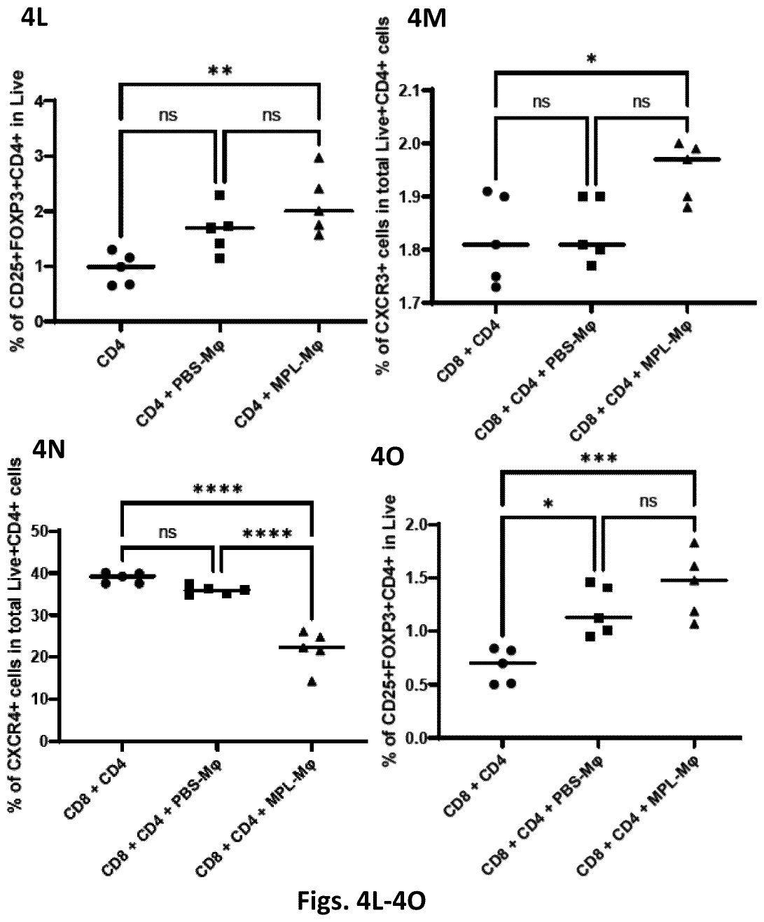

Given our observations that MPL significantly altered the infiltration and polarization of macrophages, CD4, and CD8 T cells within the irradiated tumor immune microenvironment, we sought to determine the direct effects of MPL on these populations. We cultured bone marrow derived macrophages in the presence of increasing amounts of MPL and using qPCR quantified the expression of Arg1 and Nos2, markers of M2 and M1 macrophages, respectively (59). We observed dose dependent increases in both Arg1 and Nos2, with significantly greater expression of Nos2 than Arg1 at the 100 and 200 ng/ml doses, strongly favoring M1 polarization (FIG. 4A). We observed similar trends with MPL favoring expression of pro-inflammatory cytokines (FIG. 4B) compared to anti-inflammatory cytokines (FIG. 4C).

We then tested whether in vitro MPL treatment can directly polarize naïve splenic CD4 T cells. Using Cxcr3 and Cxcr4 as markers of Th1 and Th2 cells, respectively (60, 61), we observed minimal changes in Cxcr3 and Cxcr4 expression indicating MPL treatment does not directly influence CD4 T cell polarization (FIG. 4D). When MPL was added to CD4 and CD8 cells in culture we again observed minimal increases in expression of pro-inflammatory cytokines, suggesting MPL does not have a direct effect on T cell activation (FIGS. 4E, 4F) which is consistent with prior reports investigating LPS effects on T cell populations (54).

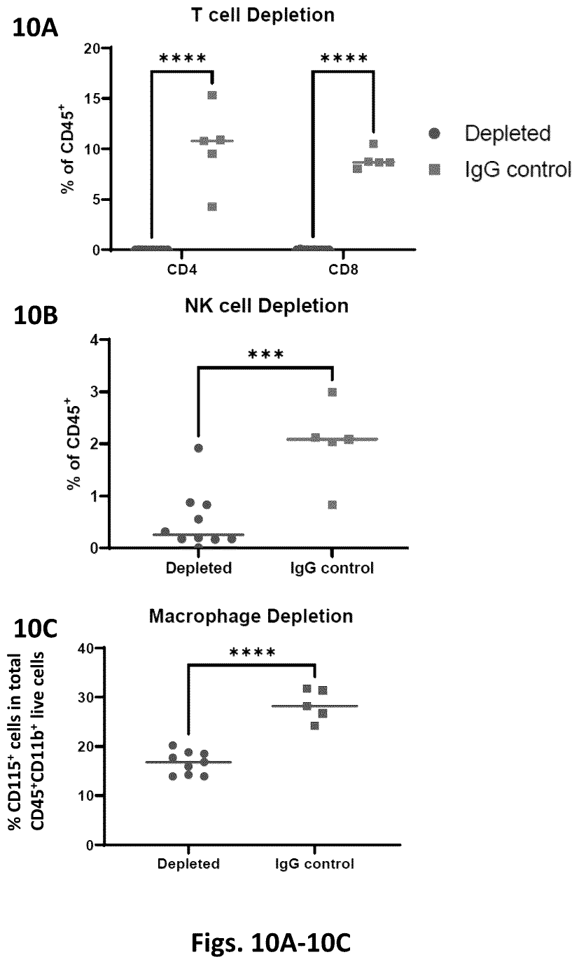

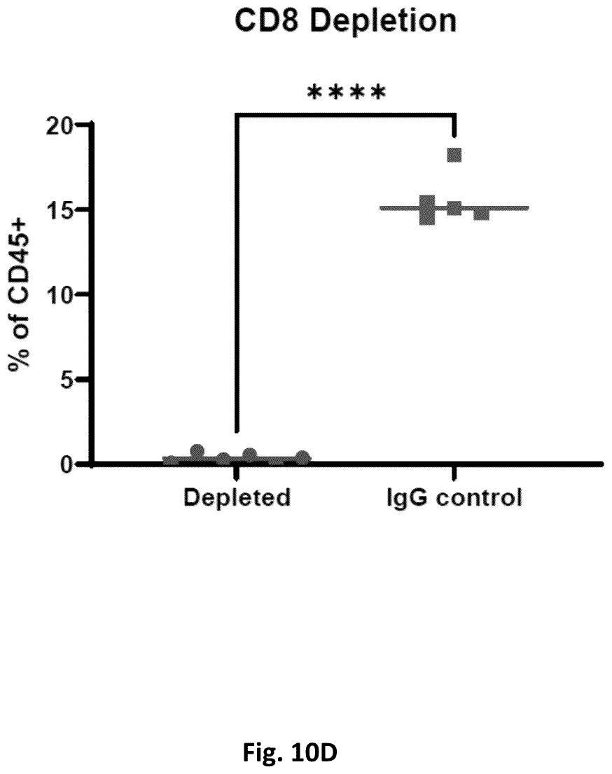

We hypothesized that MPL may favorably polarize and activate T cells through direct activation of macrophages. To test this, we co-cultured CD8 cells with macrophages stimulated with MPL or vehicle control (PBS) and quantified CD69 expression as a marker of activation. We observed that macrophages stimulated with MPL significantly increased expression of CD69 on CD8 T cells compared to CD8 T cells in monoculture or CD8 T cells cultured with unstimulated macrophages (FIG. 4H). Interestingly, when CD4 cells were added to the culture, we no longer observed an increase in CD69 expression on the CD8 cells (FIG. 4I). To investigate CD4 polarization we co-cultured CD4 cells with macrophages stimulated with MPL or PBS. We observed a significant increase in CXCR3 expression and decrease in CXCR4 expression in CD4 cells co-cultured with stimulated macrophages (FIGS. 4J, 4K). Interestingly, we also observed a significant increase in the percentage of regulatory T cells (CD4+CD25+FOXP3+) when CD4 T cells were cultured with activated macrophages (FIG. 4L). This may partially explain the loss of CD69 expression in CD8 T cells when CD4 T cells were added to the co-culture. We observed similar trends in CXCR3 and CXCR4 expression on CD4+cells, as well as increase in regulatory T cell percentage when CD8 T cells were added to the co-culture (FIGS. 4M-40).

Radiation and Anti-Tumor Antibodies Synergize with MPL Treatment to Activate Macrophages

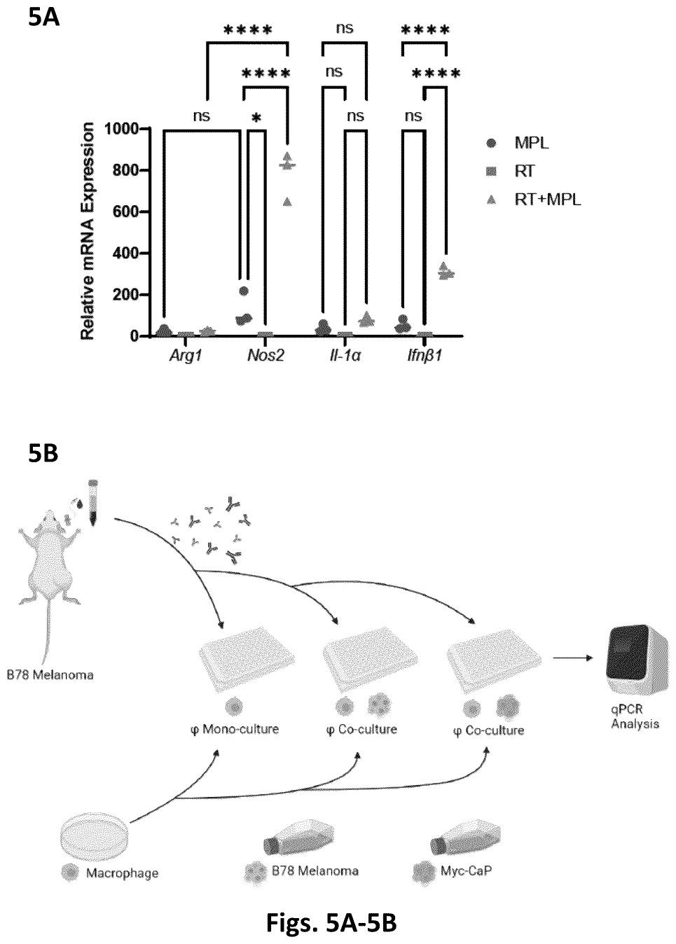

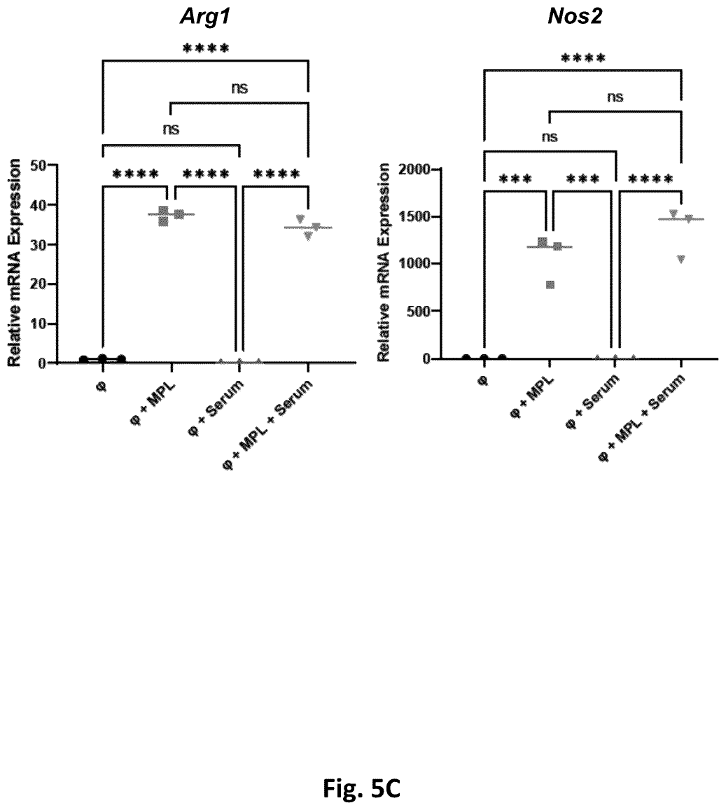

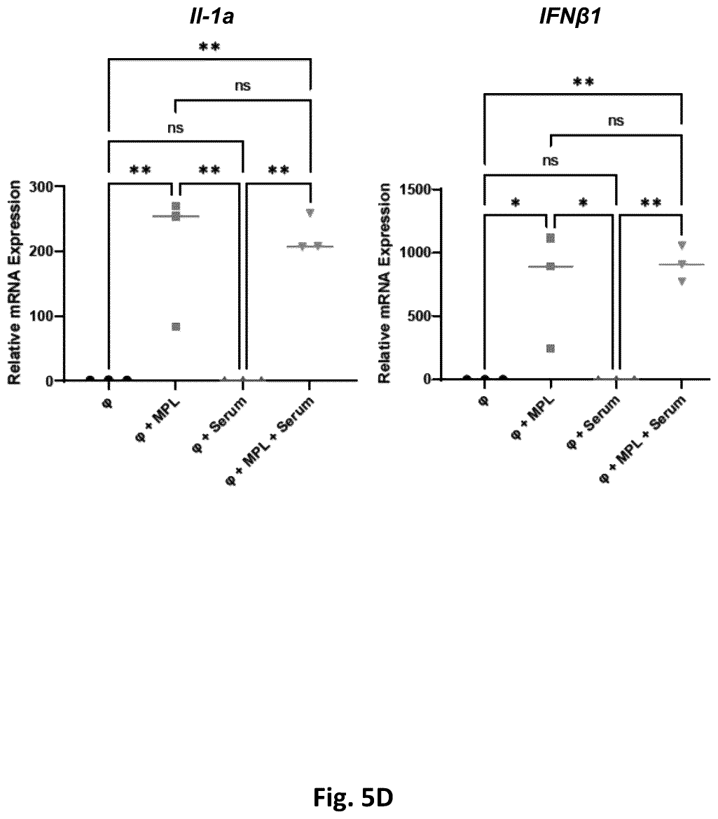

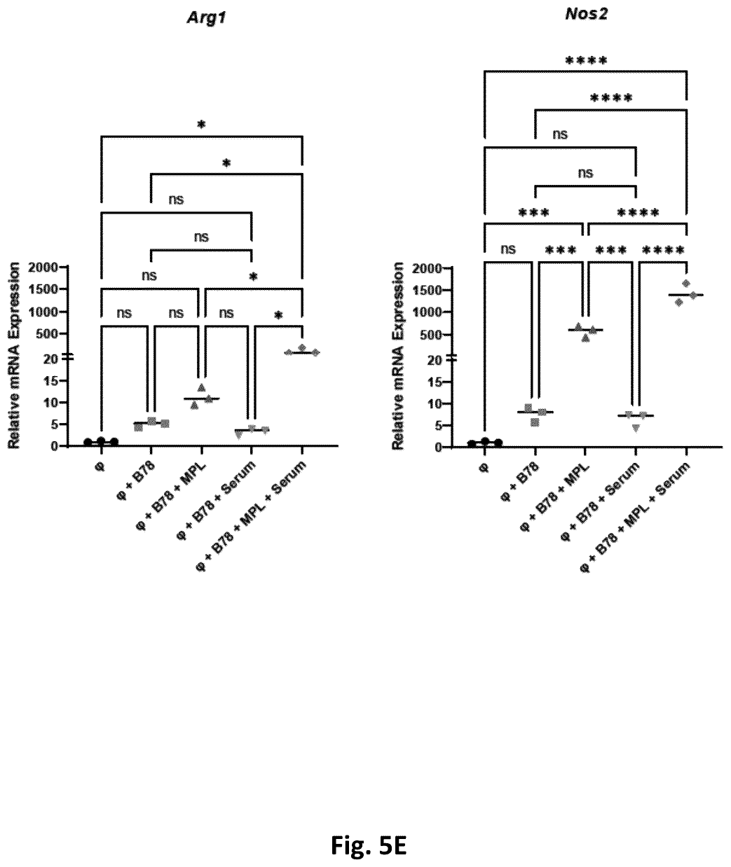

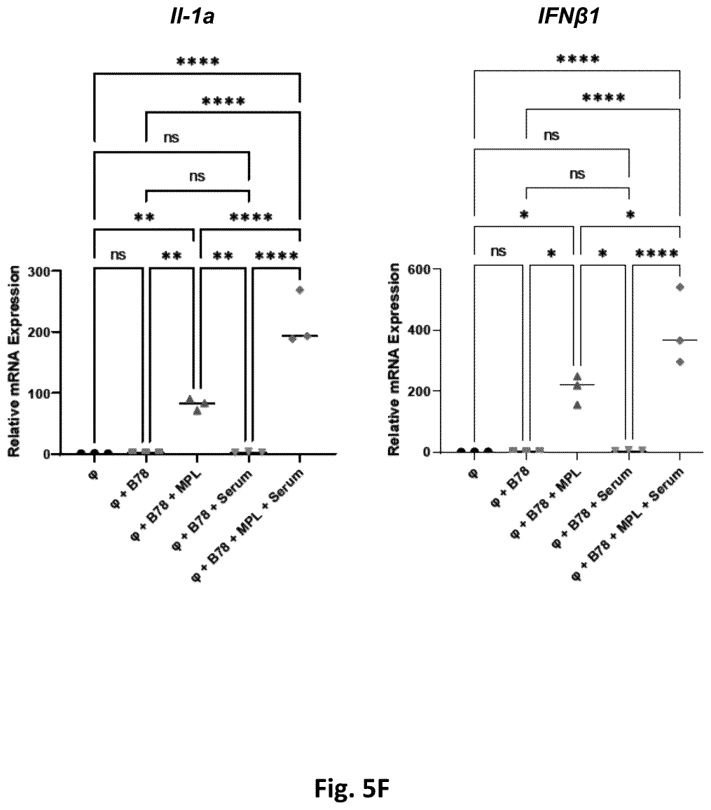

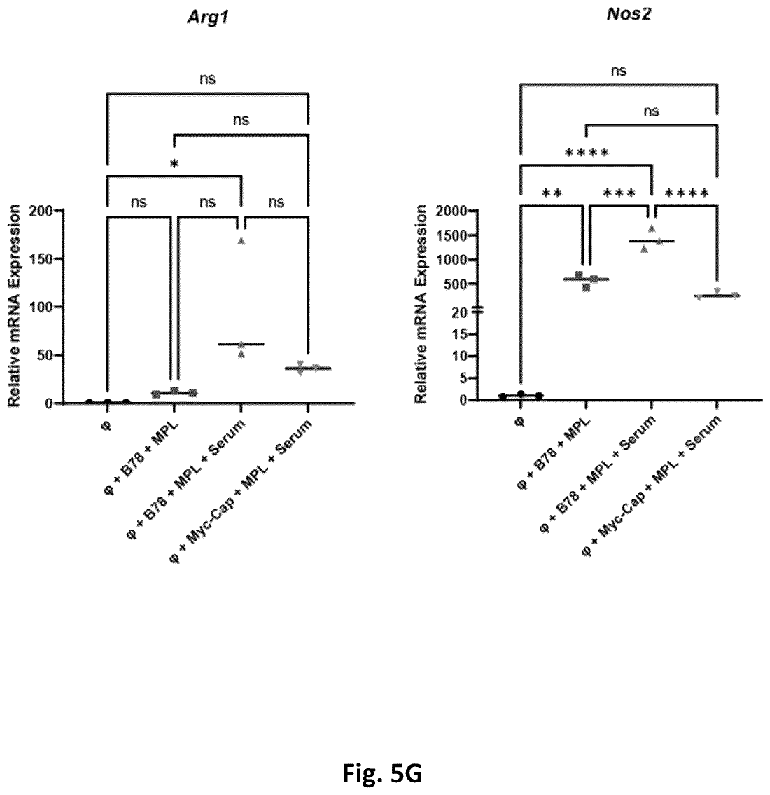

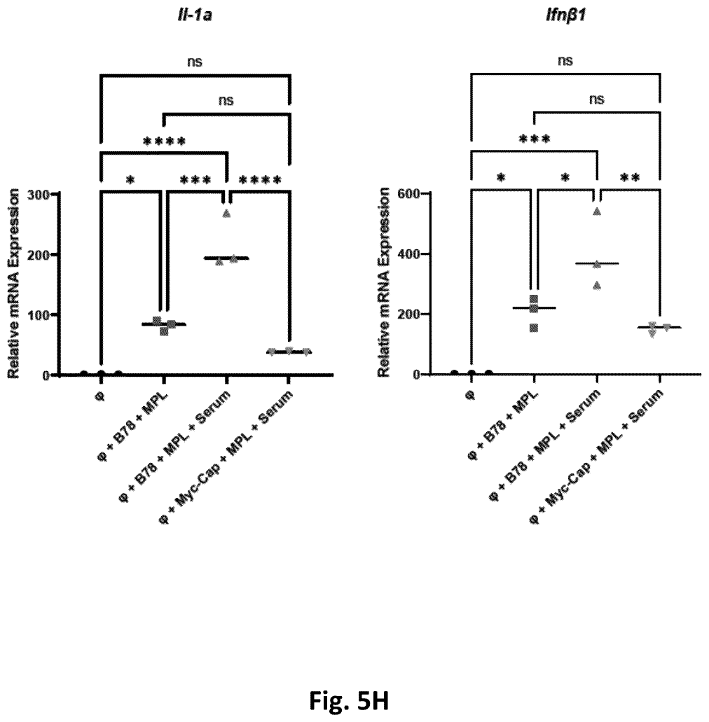

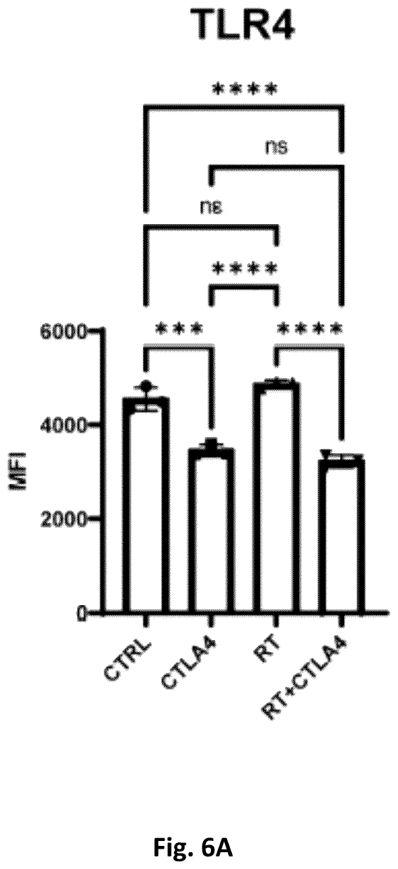

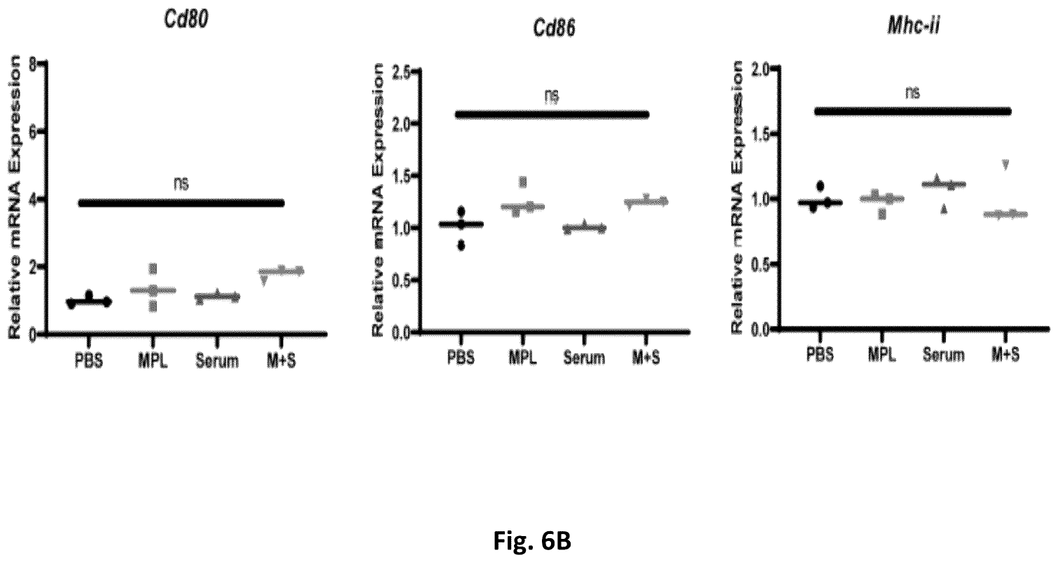

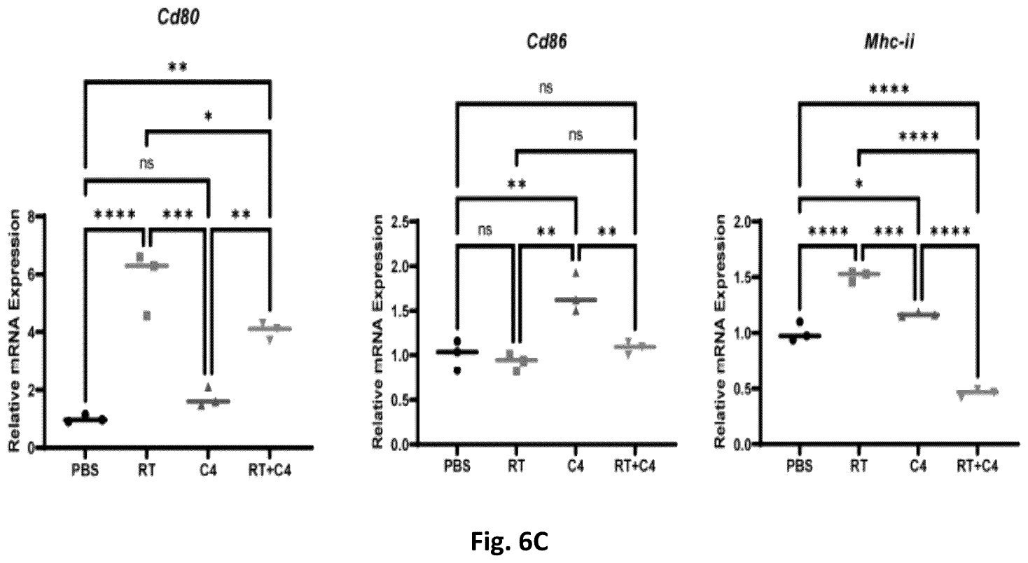

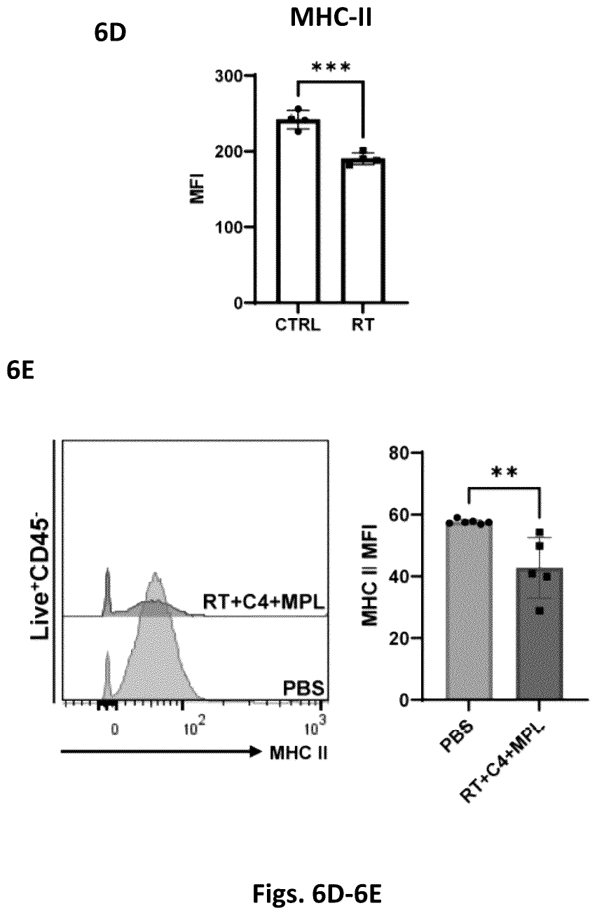

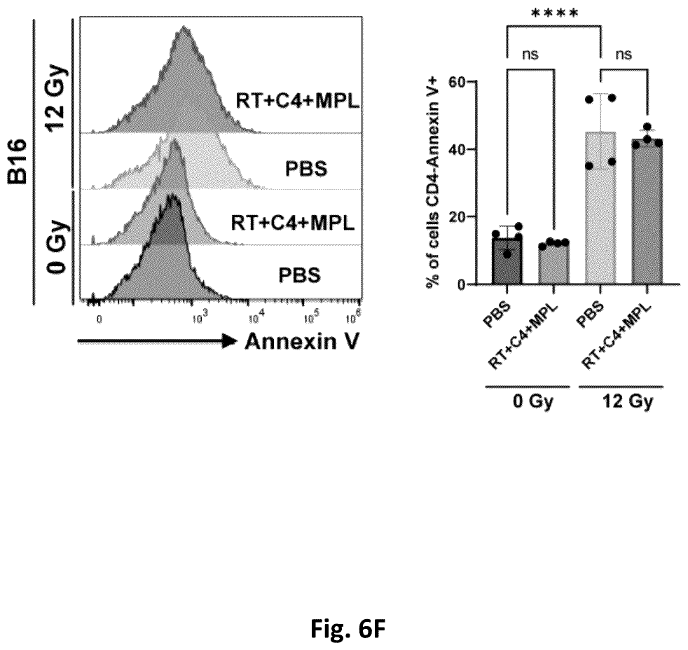

Macrophages are one of the least sensitive immune cell populations to radiation induced cell death (62). Tumor-associated macrophage populations that receive radiation in this regimen likely survive and are subsequently exposed to MPL delivered intratumorally as part of our treatment strategy. To test whether RT can synergize with MPL to further increase activation of macrophages, we delivered 12 Gy of RT to macrophages in culture and immediately replaced the growth media with fresh media containing 100 ng/mL of MPL, and 24 hours later harvested cells for analysis. We observed that the addition of RT to MPL further increased the expression of pro-inflammatory marker Ifnβ1 as well as M1 marker Nos2 compared to MPL treatment alone (FIG. 5A). Interestingly, RT had no effect on expression of TLR4 on macrophages, suggesting the enhanced activation extends beyond MPL-TLR4 receptor binding (FIG. 6A).

Given our observations that MPL can activate and favorably polarize macrophages, CD8, and CD4 T cells, we next sought to determine whether the anti-tumor antibodies generated via combination RT+C4+MPL contributed to the observed immune cell activation or functioned solely as a predictive biomarker of CR. We harvested serum on day 30 following RT from mice bearing B78 melanoma tumors that were rendered disease free following combination RT+C4+MPL treatment. We cultured bone marrow derived macrophages, alone (mono) or in co culture with B78 cells, in the presence of MPL and/or serum from mice bearing B78 tumors that were rendered disease-free following RT+MPL+C4 (FIG. 5B). In macrophages grown in monoculture, we again observed significant increases in Arg1 and Nos2 expression with the addition of MPL heavily favoring Nos2 expression. The addition of serum did not significantly increase expression of either gene, nor did the addition of serum to MPL stimulated macrophages further increase expression compared to MPL stimulation alone (FIG. 5C). Similar trends were observed with activation markers Il-1a and Ifnβ1 (FIG. 5D). When macrophages were co-cultured with B78 cells we observed a significant increase in expression of Nos2 but not Arg1 following MPL treatment. Treatment with serum alone did not significantly increase expression of either gene. However, when serum was added to macrophages stimulated with MPL we observed further significant increases in expression of both Arg1 and Nos2 with the addition of MPL favoring Nos2 expression (FIG. 5E). Similar trends to those seen with Nos2 were observed with activation markers Il-1a and Ifnβ1 (FIG. 5F). This suggests that anti-tumor antibodies can further increase activation of macrophages stimulated with MPL, but only in the presence of tumor cells. We confirmed that the effects of serum in this co-culture experiment were tumor cell specific using a control study in which macrophages were co-cultured with Myc-CaP cells that are unrelated to the B78 tumors that had been eradicated by RT+MPL+C4 in mice from whom serum was drawn. When macrophages were co-cultured with Myc-CaP cells and serum from mice rendered disease free from a B78 melanoma tumor by RT+MPL+C4, we observed no increase in the expression of macrophage polarization or activation markers with the addition of serum compared to MPL treatment alone (FIGS. 5G, 5H).

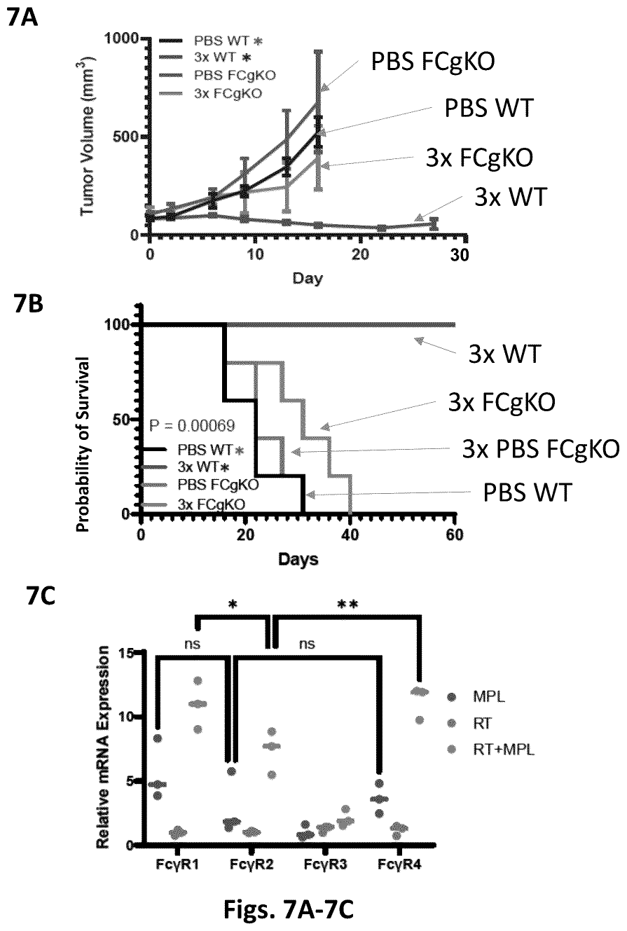

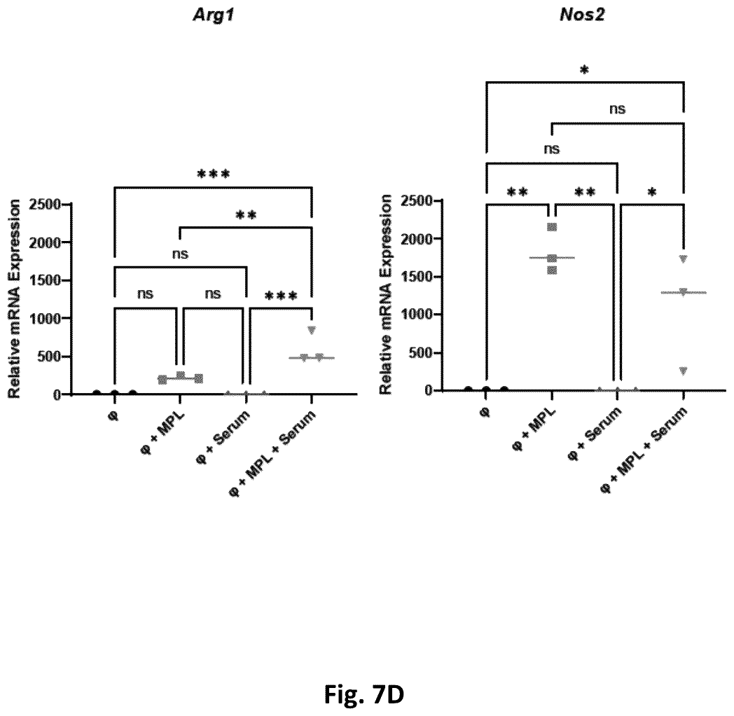

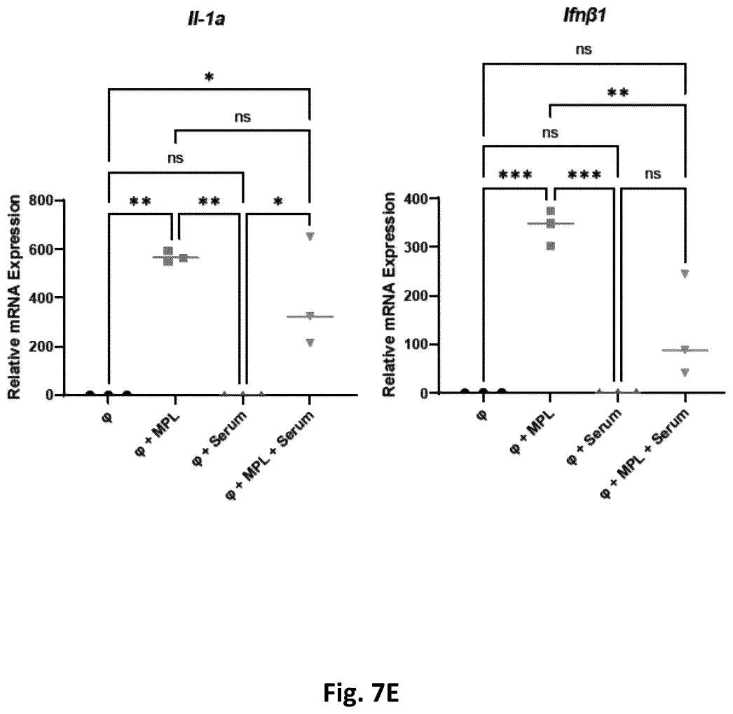

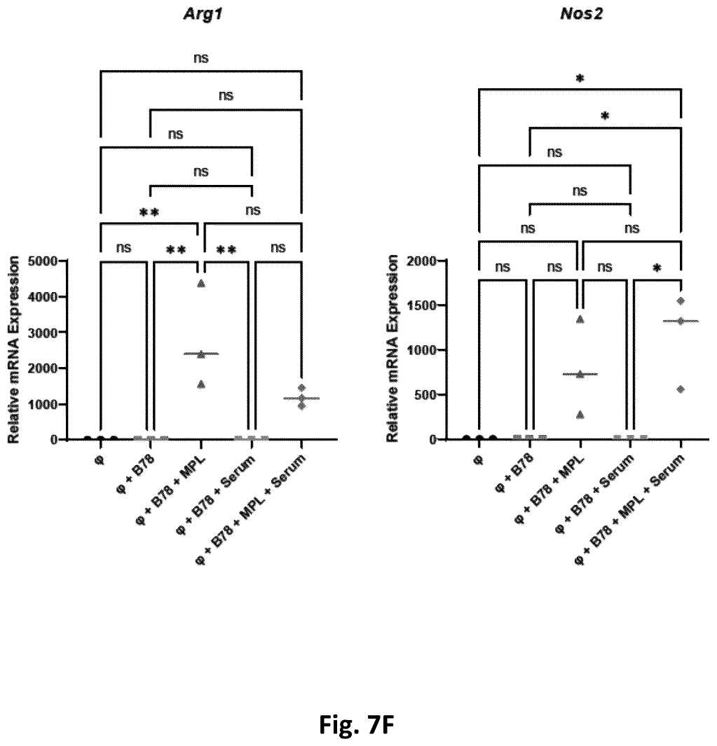

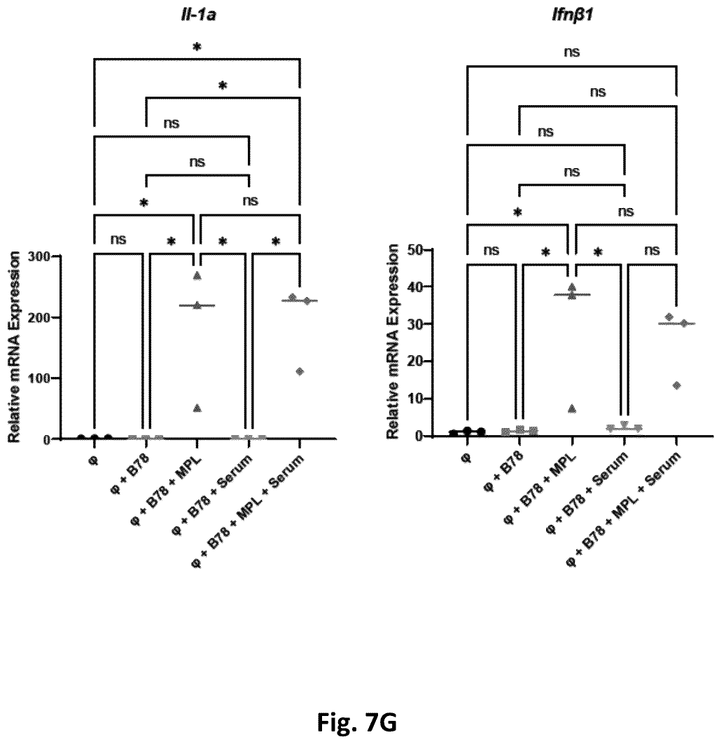

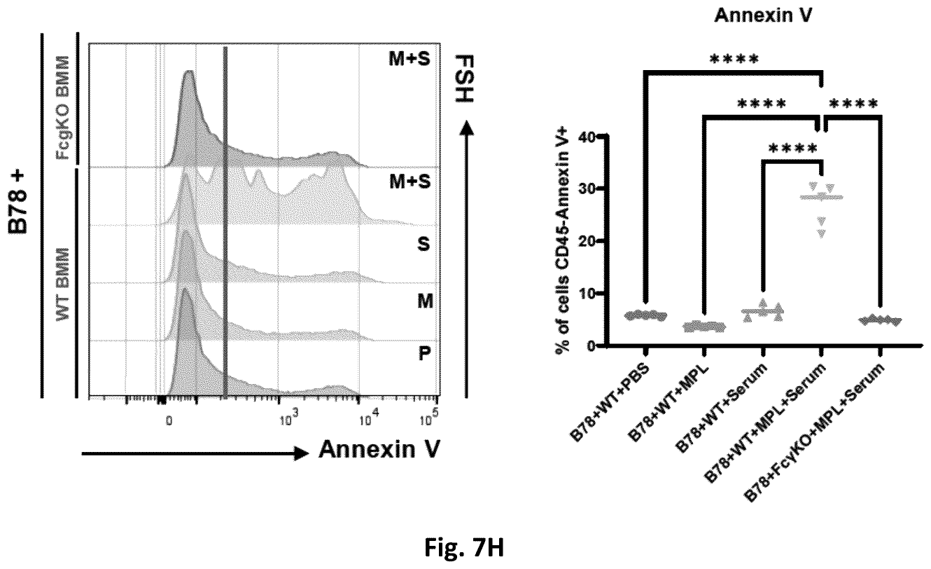

Given that serum only had activating effects in the context of MPL treatment, we hypothesized that MPL enhances binding and recognition of anti-tumor antibodies bound to tumor cells. To test this we first compared the anti-tumor efficacy of combination RT+C4+MPL in wild type and FcγR −/− mice. FcγR deficiency abrogated the anti-tumor response and survival benefit of RT+C4+MPL in FcγR −/− mice compared to wild type mice (FIGS. 7A, 7B). We then further explored the effects of RT and MPL on bone marrow derived macrophages cultured in vitro. We delivered 12 Gy of RT to macrophages in culture and immediately replaced the growth media with fresh media containing 100 ng/mL of MPL, and 24 hours later harvested cells for analysis. We observed that MPL treatment significantly increased expression of activating FcγR1 and FcγR4 as well as inhibitory FcγR2. The addition of radiation to the MPL further increased expression of these Fcγ receptors with FcγR1 and FcγR4 significantly increased compared to the increase seen for FcγR2 (FIG. 7C). To confirm that the observed synergistic activation of macrophages with MPL and serum relied on FcγR expression, we cultured bone marrow derived macrophages deficient in Fcγ receptor in mono and co-culture with B78 cells in the presence of MPL and/or serum. In FcγR −/− macrophages grown in monoculture, we again observed significant increases in Arg1 and Nos2 expression with the addition of MPL heavily favoring Nos2 expression. The addition of serum further increased expression of Arg1 but not Nos2, Il-1a, or Ifnβ1 (FIGS. 7D, 7E), somewhat similarly to that observed for wild type macrophages (FIG. 7D, 7E). When FcγR −/− macrophages were co-cultured with B78 cells we observed a significant increase in expression of Nos2 but not Arg1 following MPL treatment similar to what we observed with wild-type macrophages. In contrast to wild-type macrophages, the addition of serum in the co-culture with B78 cells, did not significantly increase expression of either gene, nor did the addition of serum to MPL stimulated FcγR −/− macrophages further increase expression compared to MPL stimulation alone (FIG. 7F). Similar trends were observed with activation markers Il-1a and Ifnβ1 confirming that enhanced activation of macrophages following serum treatment of B78 tumors is reliant on Fcγ receptor expression (FIG. 7G).

To test whether serum antibodies can induce macrophage-mediated antibody-dependent cellular cytotoxicity, we co-cultured macrophages with B78 cells with and without MPL and serum. Using Annexin V staining as a marker of apoptosis, we observed a 5-fold increase in Annexin V staining in B78 cells co-cultured with macrophages stimulated with MPL and serum compared to stimulation with each agent alone or non-stimulated macrophages. The observed increase in Annexin V staining was lost when macrophages were deficient in Fcγ receptor (FIG. 7H).

Given the functional importance of the serum antibodies, we then sought to determine the effects of our treatment regimen on B cells. We cultured B cells and treated them with either MPL, serum, MPL+serum, or PBS control. In separate experiments, we also cultured B cells and treated them with either RT, anti-CTLA-4, RT+anti-CTLA-4, or PBS control. We observed no increase in B cell activation following MPL or serum treatment and only modest activation following RT (FIGS. 6B, 6C), suggesting B cells are not directly influenced by our treatment regimen.

MPL Induced Immunity is Dependent on Th1 Cells

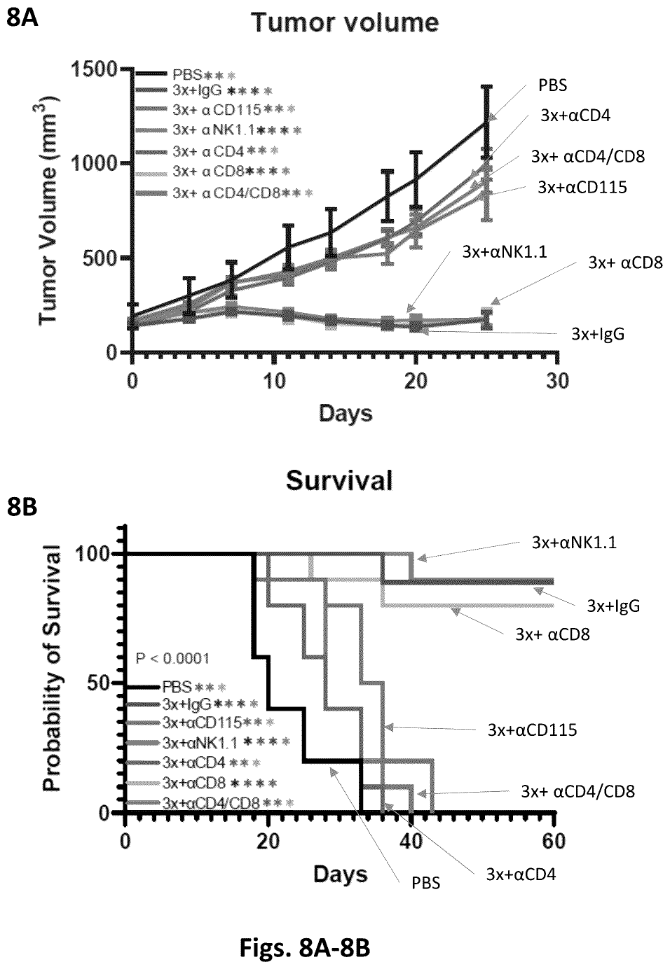

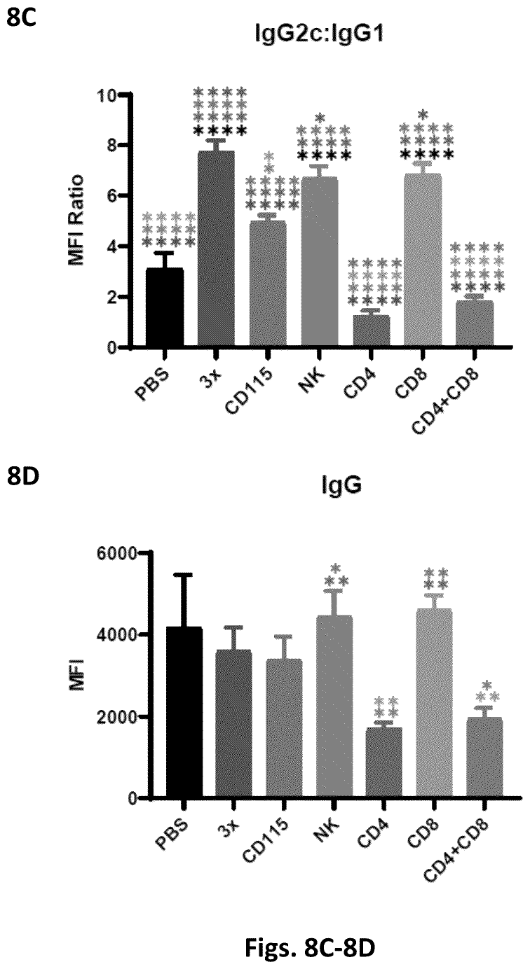

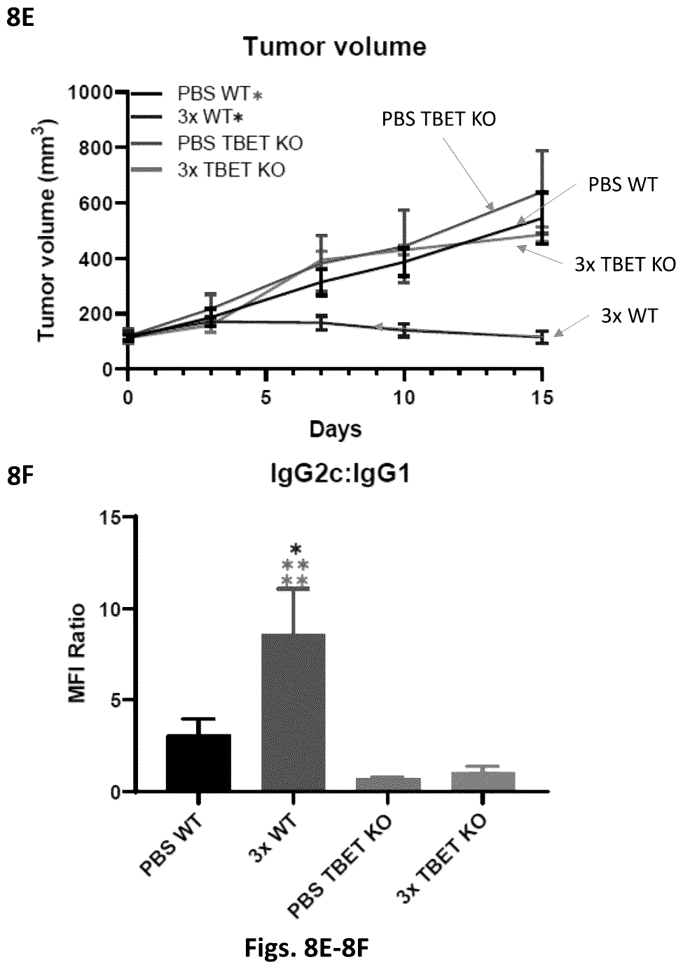

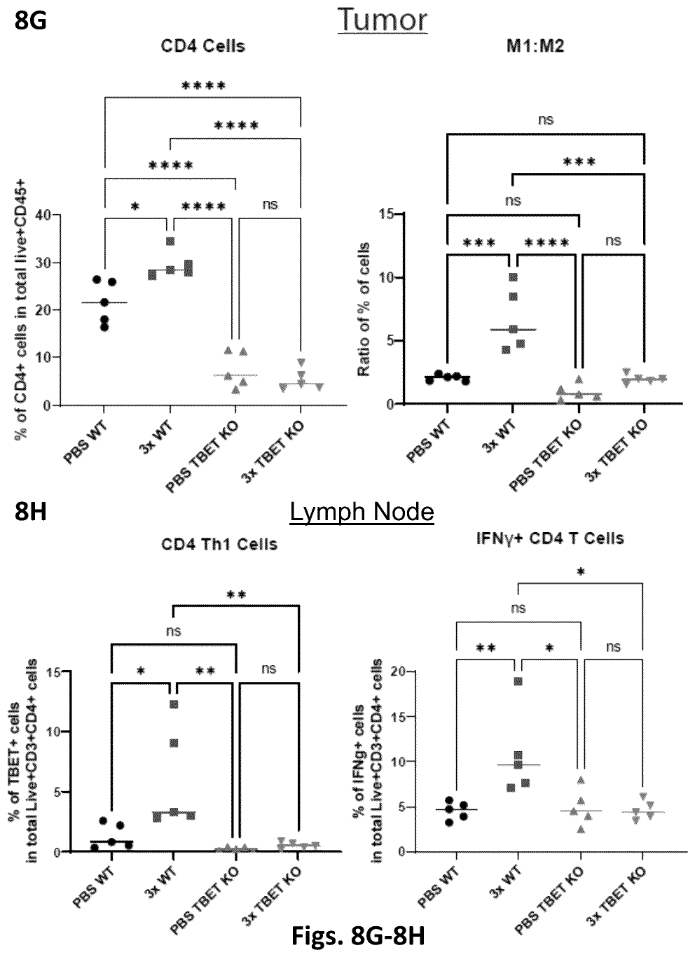

Given our observation that MPL can promote generation of anti-tumor antibodies, as well as directly activate macrophages which in turn can favor Th1 polarization of CD4 T cells and activation of CD8 T cells, we sought to determine which of these immune cells were critical for the anti-tumor response of combination RT+C4+MPL. We compared the efficacy of RT+MPL+C4 treatment in mice that were depleted of specific immune cell lineages by intraperitoneal injection of lineage-specific depleting antibodies. This included mice depleted of macrophages (αCD115), NK cells (αNK1.1), CD4 T cells (αCD4), CD8 T cells (αCD8), or both CD4 and CD8 T cells (αCD4/CD8). The loss of macrophages and CD4 cells significantly reduced the anti-tumor response compared to non-depleted mice. Interestingly, loss of NK and CD8 cells each had no effect on anti-tumor response (FIG. 8A) nor overall survival (FIG. 8B).

We collected serum at day 15 following RT for anti-tumor antibody quantification as done previously. We again observed a significant increase in the IgG2c:IgG1 ratio with combination RT+C4+MPL compared to PBS control. Depletion of NK cells or CD8 T cells had no effect on the IgG2c:IgG1 ratio, whereas macrophage depletion and CD4 T cell depletion significantly reduced the IgG2c:IgG1 ratio compared to combination RT+C4+MPL suggesting Th1 associated anti-tumor antibody class switching is dependent on both macrophages and CD4 T cells (FIG. 8C). Interestingly, depletion of CD4 T cells significantly reduced production of total IgG suggesting that anti-tumor antibody production is dependent, at least in part, on CD4 T cells (FIG. 8D).

We hypothesized that ablation of Th1 cells specifically was responsible for the loss of treatment efficacy when CD4 cells were depleted. To test this we compared the anti-tumor efficacy of combination RT+C4+MPL in wild type and TBET −/− mice. TBET deficiency abrogated the anti-tumor response and generation of Th1 associated anti-tumor antibodies following RT+C4+MPL treatment in TBET −/− mice compared to wild type mice (FIGS. 8E, 8F). We observed a significant reduction in intratumoral CD4 cell infiltration and MLM2 macrophage ratio in TBET −/− mice compared to wild type (FIG. 8G). Additionally, within the tumor draining lymph node we observed a significant reduction in Th1 C 360 D4 T cells and IFNγ+CD4 T cells in TBET −/− mice compared to wild type (FIG. 8H). Taken together, these data suggest Th1 CD4 T cells are central to the mechanism underlying generation of anti-tumor immunity with combination RT+C4+MPL.

MPL Treatment Promotes Systemic Immunity Independent of CD8 T Cells

Given that loss of CD8 cells had no effect on the anti-tumor response, we sought to determine whether CD8 cells were required for generation of systemic immunity. We used a systemic disease model consisting of a B78 primary tumor as well as intravenously injected B16 melanoma cells to model heterogeneous metastatic disease. B16 cells are parental to B78 and share common tumor neo-antigens that can be recognized by T cells (12, 54, 63). Treatment with RT+MPL+C4 significantly reduced growth at the primary tumor and significantly increased survival compared to RT+C4 (FIGS. 9A, 9B). Immediately following death or at day 60, lungs were collected to determine metastatic burden. The addition of MPL significantly reduced lung metastatic burden compared to RT+C4 (FIG. 9C). Interestingly, the enhanced anti-tumor response, survival, and decreased lung metastasis burden observed with RT+C4+MPL was independent of CD8 T cells (FIGS. 9A-9C).

Recent evidence suggests that CD4 cells may directly kill tumor cells through MHC-II mediated recognition (73). We measured expression of MHC-II on B16 tumor cells in vitro following RT as well as in vivo following combination RT+C4+MPL and in either case observed a downregulation of MHC-II expression compared with PBS control (FIGS. 6D, 6E). In addition, we isolated CD4 cells from B16 tumor bearing mice at day 15 following RT+C4+MPL treatment and co-cultured them with B16 melanoma cells. We observed no change in tumor cell killing compared with CD4 cells isolated from tumor bearing mice treated with PBS control (FIG. 6F). Together, these data suggest macrophages are the primary cytotoxic cell activated by combination treatment.

### Discussion

We have demonstrated that intratumoral injection of the vaccine adjuvant MPL can augment the anti-tumor immune response generated by RT and thereby augment response to anti-CTLA-4 checkpoint blockade. This resulted from favorable effects of MPL on polarization of both M1 macrophages and Th1 CD4 T cells in the radiated tumor microenvironment. Consistent with Th1 CD4 T cell polarization, we observed that MPL induced production of IgG2c dominant anti-tumor antibodies as well as upregulation of the IgG2c high-affinity Fcγ receptors I and IV on macrophages. MPL-stimulated macrophages exposed to anti-tumor antibodies on tumor cells exhibited increased activation and direct killing of tumor cells in vitro. Depletion of macrophages, Th1 CD4 T cells, or loss of Fcγ receptor expression completely abrogated the anti-tumor immune response in vivo. Lastly, we demonstrate the capacity of our combination treatment regimen to generate local and systemic immune responses through a CD8 T cell independent mechanism.

CD8 T cells have classically been thought of as the “gold standard” in immunotherapy for potentiating tumor eradication and generating immunologic memory. However, recent studies have suggested the importance of other cell populations in generating anti-tumor immunity. Natural killer cells may mediate anti-tumor immunity through antibody dependent cell-mediated cytotoxicity (64, 65). Vaccine based immunotherapy strategies aim to stimulate B cell mediated production of tumor specific antibodies that can bind to tumor antigens in the blood or at the tumor site. Antibody binding can enable a variety of responses including neutralization of the target protein function (66), direct tumor clearance via phagocytosis (67), complement dependent cytotoxicity (68), or antibody dependent cell-mediated cytotoxicity (69). In addition to helper functions, CD4 T cells have been shown to have direct cytotoxic activity in preclinical models (70-73) which has been confirmed clinically in melanoma (74) and hepatocellular carcinoma (75).

Here we report generation of a CD8 independent systemic immune response that is dependent on both Th1 CD4 T cells and macrophages. Our data suggest that CD4 T cell polarization towards a Th1 phenotype is required to generate functional anti-tumor antibodies that are IgG2c subclass dominant. We demonstrate that these antibodies have the potential to enable macrophage mediated tumor cell killing in vitro and in vivo but only when macrophages have been stimulated with MPL. Interestingly, loss of natural killer cells in vivo did not abrogate the anti-tumor immune response despite their ability to generate antibody dependent cell mediated cytotoxicity. This may be at least partially explained by the observation that NK cells express relatively low amounts of TLR4 compared to macrophages (76), and that based on our data, functional antibody recognition in this model system requires MPL stimulation.

Prior studies have suggested potential benefit of combining MPL with checkpoint blockade. In preclinical models MPL has been shown to increase the anti-tumor efficacy of anti-PD-1/anti-PD-L1 therapy through activation of dendritic cells and enhanced antigen presentation (77, 78). Within the tumor draining lymph node we observed a significant increase in dendritic cell activation following combination radiation and anti-CTLA-4, which is consistent with prior reports (7, 38, 39). However, the addition of MPL did not further increase dendritic cell activation, suggesting that the role of MPL in our treatment regimen extends beyond supporting antigen presentation.

As a general class of immune adjuvants, TLR4 agonists have gained interest in testing their potential to enhance conventional vaccine efficacy, namely through promotion of Th1 polarization (79-81). In our model, we observed a significant increase in Th1 polarization when MPL was added to RT and anti-CTLA-4, but not as a single agent. Interestingly, the depth of response to treatment positively correlated with magnitude of Th1 polarization as measured by IgG2c class switching. The magnitude of IgG2c class switching was significantly increased in mice developing a complete response, but only when MPL was added to RT and anti-CTLA-4. This is in contrast to prior reports demonstrating that anti-CTLA-4 enables expansion of Th1-like CD4 T cell populations (82, 83). However, it is notable that these studies were conducted in the MC-38 colon carcinoma model, which possesses relatively high immunogenicity. Additionally, the authors confirmed these findings in the poorly immunogenic B16 melanoma model, which is the parental cell line to our B78 melanoma model, but required treatment with the GVAX tumor vaccine in order to boost overall T cell infiltration to enable their analyses. This overall lack of T cell infiltration could at least partially explain the lack of observed Th1 polarization with combination RT and anti-CTLA-4 in our models.

In addition to serving as a potential biomarker of response to treatment, Th1 anti-tumor antibody class switching served a functional purpose and was critical for response to combination treatment in our model through Fcγ receptor mediated recognition. Prior reports have described the importance of FcγR4 in mediating anti-CTLA-4 induced regulatory T cell depletion (84) which can be enhanced by upregulation of FcγR4 through TLR1/2 agonist treatment (85). These findings build on similar reports describing Fc receptor subclass importance in the context of monoclonal antibody treatment (86-88).

However, to our knowledge, this is the first report describing the importance of endogenously generated anti-tumor antibody class dominance and recognition through macrophage Fcγ receptor binding in generating a successful anti-tumor immune response. Four individual Fcγ receptors have been identified in mice, with all four being expressed on macrophages. The receptors FcγR1 and FcγR4 have a high affinity for the antibody subclass IgG2c only, whereas FcγR2 and FcγR3 have a low affinity for both IgG2c and IgG1 (89). Given our combination treatment results in antibody populations that significantly favor IgG2c over IgG1, these anti-tumor antibodies selectively stimulate the activating receptors FcγR1 and FcγR4, of which the expression of these are increased with MPL treatment, and further increased with the addition of RT. These mechanisms may underlie the observed synergy between MPL and combination RT and anti-CTLA-4.

We acknowledge several limitations of this study which include the use of syngeneic heterotopic murine tumor models that may not fully recapitulate the tumor heterogeneity nor the immune microenvironment that is observed in humans. Although our findings in two separate syngeneic tumor models of melanoma and prostate cancer suggest that the addition of MPL to combination RT and anti-CTLA-4 is a promising treatment strategy, others have previously shown that syngeneic tumor models can possess pre-existing immunity that is critical for the response to RT and immune checkpoint inhibition (90, 91). Therefore, additional studies with this combination treatment strategy in spontaneously developing murine tumor models would further support the potential for successful clinical translation. A similar limitation arises from the observation that anti-CTLA-4 generates superior immune responses compared with anti-PD-1/L1 therapies in many murine tumor models, likely due, at least in part, to regulatory T cell depletion generated via CTLA-4 blockade. This directly contrasts with clinical studies and further highlights key differences between murine and human immunity. To overcome this limitation and test whether MPL may enhance the antitumor immune response to combination RT and anti-CTLA-4, we have focused our efforts on utilizing poorly immunogenic murine tumor models that do not respond well to anti-CTLA-4 monotherapy. For RT treatment, we used a single fraction of 12 Gy based on our previous data in the B78 tumor model, however, we did not test other RT doses or fractionation schemes of which may influence treatment efficacy. Future work dedicated to testing MPL in combination with other RT modalities, doses, and fractionation schemes will help fully determine the translational potential of MPL treatment in the context of RT. Lastly, our treatment strategy involves the intratumoral administration of MPL which may limit translational potential. However, several immunotherapy regimens are currently in development that use intratumoral approaches to delivery (92). In addition, advances in image guidance within the field of interventional radiology may enable translation to a broad range of cancer types.

There is rapidly growing interest in developing in situ vaccination strategies that incorporate radiation therapy. To date there are nearly 600 active trials that are investigating radiation in combination with anti-CTLA-4 or PD-1/L1 checkpoint blockade (93). In addition to checkpoint blockade, a variety of other immunotherapies are under investigation including cytokines such as IL-2 and IL-15, cell-based therapies such as CAR-T cells, cancer vaccines, oncolytic viruses, and antibody agonists such as anti-OX40 and anti-GITR. Each of these in situ vaccination strategies may benefit from combination with additional adjuvants such as MPL, and further preclinical and clinical studies are warranted to fully investigate the potential of combination therapy strategies.

### REFERENCES

1. J. Bernier, et al., Radiation oncology: a century of achievements. Nat Rev Cancer 4, 737-747 (2004).
2. E. B. Golden, et al., Radiation fosters dose-dependent and chemotherapy-induced immunogenic cell death. Oncoimmunology 3, e28518 (2014).
3. E. B. Golden, et al., Radiotherapy and immunogenic cell death. Semin Radiat Oncol 25, 11-17 (2015).
4. S. Demaria, et al., Combining radiotherapy and immunotherapy: a revived partnership. Int J Radiat Oncol Biol Phys 63, 655-666 (2005).
5. L. R. Werner, et al., Transcriptional-mediated effects of radiation on the expression of immune susceptibility markers in melanoma. Radiother Oncol 124, 418-426 (2017).
6. L. Deng, et al., STING-Dependent Cytosolic DNA Sensing Promotes Radiation-Induced Type I Interferon-Dependent Antitumor Immunity in Immunogenic Tumors. Immunity 41, 843-852 (2014).
7. C. Vanpouille-Box, et al., DNA exonuclease Trex1 regulates radiotherapy-induced tumour immunogenicity. Nat Commun 8, 15618 (2017).
8. J. C. Jagodinsky, et al., Temporal analysis of type 1 interferon activation in tumor cells following external beam radiotherapy or targeted radionuclide therapy. Theranostics 11, 6120-6137 (2021).
9. S. Z. Liu, Nonlinear dose-response relationship in the immune system following exposure to ionizing radiation: mechanisms and implications. Nonlinearity Biol Toxicol Med 1, 71-92 (2003).
10. M. E. Rodriguez-Ruiz, et al., Intercellular Adhesion Molecule-1 and Vascular Cell Adhesion Molecule Are Induced by Ionizing Radiation on Lymphatic Endothelium. Int J Radiat Oncol Biol Phys 97, 389-400 (2017).
11. T. A. Waldmann, Cytokines in Cancer Immunotherapy. Cold Spring Harb Perspect Biol 10, (2018).
12. Z. S. Morris, et al., In Situ Tumor Vaccination by Combining Local Radiation and Tumor-Specific Antibody or Immunocytokine Treatments. Cancer Res 76, 3929-3941 (2016).
13. A. Marabelle, et al., Intratumoral immunotherapy: using the tumor as the remedy. Ann Oncol 28, xii33-xii43 (2017).
14. R. H. Pierce, et al., In-situ tumor vaccination: Bringing the fight to the tumor. Hum Vaccin Immunother 11, 1901-1909 (2015).
15. B. B. Campbell, et al., Comprehensive Analysis of Hypermutation in Human Cancer. Cell 171, 1042-1056 e1010 (2017).
16. S. C. Formenti, et al., Radiotherapy induces responses of lung cancer to CTLA-4 721 blockade. Nat Med 24, 1845-1851 (2018).
17. Z. G. Fridlender, et al., Polarization of tumor-associated neutrophil phenotype by TGF-beta: “N1” versus “N2” TAN. Cancer Cell 16, 183-194 (2009).
18. E. L. Kachikwu, et al., Radiation enhances regulatory T cell representation. Int J Radiat Oncol Biol Phys 81, 1128-1135 (2011).
19. H. Tanaka, et al., Transforming growth factor beta signaling inhibitor, SB-431542, induces maturation of dendritic cells and enhances anti-tumor activity. Oncol Rep 24, 1637-1643 (2010).
20. C. S. Tsai, et al., Macrophages from irradiated tumors express higher levels of iNOS, arginase-I and COX-2, and promote tumor growth. Int J Radiat Oncol Biol Phys 68, 499-507 (2007).
21. J. Xu, et al., CSF1R signaling blockade stanches tumor-infiltrating myeloid cells and improves the efficacy of radiotherapy in prostate cancer. Cancer Res 73, 2782-2794 (2013).
22. Y. Abuodeh, et al., Systematic review of case reports on the abscopal effect. Curr Probl Cancer 40, 25-37 (2016).
23. A. J. Korman, et al., Checkpoint blockade in cancer immunotherapy. Adv Immunol 90, 297-339 (2006).
24. M. Binnewies, et al., Understanding the tumor immune microenvironment (TIME) for effective therapy. Nat Med 24, 541-550 (2018).
25. R. Cristescu, et al., Pan-tumor genomic biomarkers for PD-1 checkpoint blockade-based immunotherapy. Science 362, (2018).
26. T. F. Gajewski et al., Cancer Immunotherapy Targets Based on Understanding the T Cell-Inflamed Versus Non-T Cell-Inflamed Tumor Microenvironment. Adv Exp Med Biol 1036, 19-31 (2017).
27. J. Galon, et al., Approaches to treat immune hot, altered and cold tumours with combination immunotherapies. Nat Rev Drug Discov 18, 197-218 (2019).
28. C.-S. V. Hodi F S, et al., Nivolumab plus ipilimumab or nivolumab alone versus ipilimumab alone in advanced melanoma (CheckMate 067): 4-year outcomes of a multicentre, randomised, phase 3 trial. Lancet Oncol 19, (2018).
29. J. N. Kather, et al., Topography of cancer-associated immune cells in human solid tumors. Elife 7, (2018).
30. D. Lindau, et al., The immunosuppressive tumour network: myeloid-derived suppressor cells, regulatory T cells and natural killer T cells. Immunology 138, 105-115 (2013).
31. T. M. Beer, et al., Randomized, Double-Blind, Phase III Trial of Ipilimumab Versus Placebo in Asymptomatic or Minimally Symptomatic Patients With Metastatic Chemotherapy-Naive Castration-Resistant Prostate Cancer. J Clin Oncol 35, 40-47 (2017).
32. E. D. Kwon, et al., Ipilimumab versus placebo after radiotherapy in patients with metastatic castration-resistant prostate cancer that had progressed after docetaxel chemotherapy (CA184-043): a multicentre, randomised, double-blind, phase 3 trial. Lancet Oncol 15, 700-712 (2014).
33. M. Y. Mapara, et al., Tolerance and cancer: mechanisms of tumor evasion and strategies for breaking tolerance. J Clin Oncol 22, 1136-1151 (2004).
34. L. Deng, H et al., Irradiation and anti-PD-L1 treatment synergistically promote antitumor immunity in mice. J Clin Invest 124, 687-695 (2014).
35. L. Deng, et al., Radiation and anti-PD-L1 antibody combinatorial therapy induces T cell-mediated depletion of myeloid-derived suppressor cells and tumor regression. Oncoimmunology 3, e28499 (2014).
36. C. Twyman-Saint Victor, et al., Radiation and dual checkpoint blockade activate non-redundant immune mechanisms in cancer. Nature 520, 373-377 (2015).
37. S. Demaria, et al., Ionizing radiation inhibition of distant untreated tumors (abscopal effect) is immune mediated. Int J Radiat Oncol Biol Phys 58, 862-870 (2004).
38. S. Demaria, et al., Immune792 mediated inhibition of metastases after treatment with local radiation and CTLA-4 blockade in a mouse model of breast cancer. Clin Cancer Res 11, 728-734 (2005).
39. M. Z. Dewan, et al., Fractionated but not single-dose radiotherapy induces an immune-mediated abscopal effect when combined with anti-CTLA-4 antibody. Clin Cancer Res 15, 5379-5388 (2009).
40. N. P. Rudqvist, et al., Radiotherapy and CTLA-4 Blockade Shape the TCR Repertoire of Tumor-Infiltrating T Cells. Cancer Immunol Res 6, 139-150 (2018).
41. J. M. Diamond, et al., Exosomes Shuttle TREX1-Sensitive IFN-Stimulatory dsDNA from Irradiated Cancer Cells to DCs. Cancer Immunol Res 6, 910-920 (2018).
42. S. C. Formenti, et al., Systemic effects of local radiotherapy. Lancet Oncol 10, 718-726 (2009).
43. S. M. McBride, et al., A phase II randomized trial of nivolumab with stereotactic body radiotherapy (SBRT) versus nivolumab alone in metastatic (M1) head and neck squamous cell carcinoma (HNSCC). Journal of Clinical Oncology 36, 6009-6009 (2018).
44. W. Theelen, et al., Randomized phase II study of pembrolizumab after stereotactic body radiotherapy (SBRT) versus pembrolizumab alone in patients with advanced non-small cell lung cancer: The PEMBRO-RT study, (2018).
45. W. Theelen, et al., Effect of Pembrolizumab After Stereotactic Body Radiotherapy vs Pembrolizumab Alone on Tumor Response in Patients With Advanced Non-Small Cell Lung Cancer: Results of the PEMBRO-RT Phase 2 Randomized Clinical Trial. JAMA Oncol, (2019).
46. Y. Q. Wang, et al., MPL Adjuvant Contains Competitive Antagonists of Human TLR4. Front Immunol 11, 577823 (2020).
47. C. Centers for Disease, Prevention, FDA licensure of bivalent human papillomavirus vaccine (HPV2, Cervarix) for use in females and updated HPV vaccination recommendations from the Advisory Committee on Immunization Practices (ACIP). MMWR Morb Mortal Wkly Rep 59, 626-629 (2010).
48. L. B. Alexandrov, et al., Signatures of mutational processes in human cancer. Nature 500, 415-421 (2013).
49. F. S. Hodi, et al., Improved survival with ipilimumab in patients with metastatic melanoma. N Engl J Med 363, 711-723 (2010).
50. C. Robert, et al., Nivolumab in previously untreated melanoma without BRAF mutation. N Engl J Med 372, 320-330 (2015).
51. T. F. Gajewski, The Next Hurdle in Cancer Immunotherapy: Overcoming the Non-T-Cell-Inflamed Tumor Microenvironment. Semin Oncol 42, 663-671 (2015).
52. Z. S. Morris, et al., Tumor-Specific Inhibition of. Cancer Immunol Res 6, 825-834 (2018).
53. S. L. Topalian, et al., Safety, activity, and immune correlates of anti-PD-1 antibody in cancer. N Engl J Med 366, 2443-2454 (2012).
54. P. A. Clark, et al., In situ vaccination at a peripheral tumor site augments response against melanoma brain metastases. J Immunother Cancer 8, (2020).
55. C. C. Baniel, et al., In situ Vaccine Plus Checkpoint Blockade Induces Memory Humoral Response. Front Immunol 11, 1610 (2020).
56. S. Nazeri, et al., Measuring of IgG2c isotype instead of IgG2a in immunized C57BL/6 mice with Plasmodium vivax TRAP as a subunit vaccine candidate in order to correct interpretation of Th1 versus Th2 immune response. Exp Parasitol 216, 107944 (2020).
57. Y. Shi, et al., Synergy of anti-CD40, CpG and MPL in activation of mouse macrophages. Mol Immunol 66, 208-215 (2015).
58. K. De Filippo, et al., Mast cell and macrophage chemokines CXCL1/CXCL2 control the early stage of neutrophil recruitment during tissue inflammation. Blood 121, 4930-4937 (2013).
59. M. Orecchioni, et al., Macrophage Polarization: Different Gene Signatures in M1(LPS+) vs. Classically and M2(LPS−) vs. Alternatively Activated Macrophages. Front Immunol 10, 1084 (2019).
60. S. Qin, et al., The chemokine receptors CXCR3 and CCR5 mark subsets of T cells associated with certain inflammatory reactions. J Clin Invest 101, 746-754 (1998).
61. F. Sallusto, et al., Flexible programs of chemokine receptor expression on human polarized T helper 1 and 2 lymphocytes. J ExpMed 187, 875-883 (1998).
62. D. Heylmann, et al., Comparison of DNA repair and radiosensitivity of different blood cell populations. Sci Rep 11, 2478 (2021).
63. M. Haraguchi, et al., Isolation of GD3 synthase gene by expression cloning of GM3 alpha-2,8-sialyltransferase cDNA using anti-GD2 monoclonal antibody. Proc Natl Acad Sci USA 91, 10455-10459 (1994).
64. G. E. Pluhar, et al., CD8+ T Cell-Independent Immune-Mediated Mechanisms of Anti-Tumor Activity. Crit Rev Immunol 35, 153-172 (2015).
65. W. J. Jin, et al., Tumor-Specific Antibody, Cetuximab, Enhances the In Situ Vaccine Effect of Radiation in Immunologically 888 Cold Head and Neck Squamous Cell Carcinoma. Front Immunol 11, 591139 (2020).
66. R. L. Ferris, et al., Tumor antigen-targeted, monoclonal antibody-based immunotherapy: clinical response, cellular immunity, and immunoescape. J Clin Oncol 28, 4390-4399 (2010).
67. J. Uchida, et al., The innate mononuclear phagocyte network depletes B lymphocytes through Fc receptor-dependent mechanisms during anti-CD20 antibody immunotherapy. J Exp Med 199, 1659-1669 (2004).
68. A. D. Kennedy, et al., An anti-C3b(i) mAb enhances complement activation, C3b(i) deposition, and killing of CD20+cells by rituximab. Blood 101, 1071-1079 (2003).
69. K. A. Murphy, et al., CD8+ T cell-independent tumor regression induced by Fc-OX40L and therapeutic vaccination in a mouse model of glioma. J Immunol 192, 224-233 (2014).
70. H. Cheroutre, et al., CD4 CTL: living up to the challenge. Semin Immunol 25, 273-281 (2013).
71. S. K. Hildemann, et al., High efficiency of antiviral CD4(+) killer T cells. PLoS One 8, e60420 (2013).
72. S. A. Quezada, et al., Tumor-reactive CD4(+) T cells develop cytotoxic activity and eradicate large established melanoma after transfer into lymphopenic hosts. J Exp Med 207, 637-650 (2010).
73. D. Y. Oh, et al., Cytotoxic CD4(+) T cells in cancer: Expanding the immune effector toolbox. Immunity 54, 2701-2711 (2021).
74. S. Kitano, et al., Enhancement of tumor-reactive cytotoxic CD4+ T cell responses after ipilimumab treatment in four advanced melanoma patients. Cancer Immunol Res 1, 235-244 (2013).
75. J. Fu, et al., Impairment of CD4+cytotoxic T cells predicts poor survival and high recurrence rates in patients with hepatocellular carcinoma. Hepatology 58, 139-149 (2013).
76. V. Hornung, et al., Quantitative expression of toll-like receptor 1-10 mRNA in cellular subsets of human peripheral blood mononuclear cells and sensitivity to CpG oligodeoxynucleotides. J Immunol 168, 4531-4537 (2002).
77. Y. Jeong, et al., Dendritic cell activation by an E. coli-derived monophosphoryl lipid A enhances the efficacy of PD-1 blockade. Cancer Lett 472, 19-28 (2020).
78. W. Zhang, et al., Monophosphoryl lipid A-induced activation of plasmacytoid dendritic cells enhances the anti-cancer effects of anti-PD-L1 antibodies. Cancer Immunol Immunother 70, 689-700 (2021).
79. S. L. Lambert, et al., Molecular and cellular response profiles induced by the TLR4 agonist-based adjuvant Glucopyranosyl Lipid A. PLoS One 7, e51618 (2012).
80. M. T. Orr, et al., MyD88 and TRIF synergistic interaction is required for TH1-cell polarization with a synthetic TLR4 agonist adjuvant. Eur J Immunol 43, 2398-2408 (2013).
81. A. L. Desbien, et al., Squalene emulsion potentiates the adjuvant activity of the TLR4 agonist, GLA, via inflammatory caspases, IL-18, and IFN-γ. Eur J Immunol 45, 407-417 (2015).
82. S. C. Wei, et al., Distinct Cellular Mechanisms Underlie Anti-CTLA-4 and Anti-PD-1 Checkpoint Blockade. Cell 170, 1120-1133.e1117 (2017).
83. S. C. Wei, et al., Combination anti-CTLA-4 plus anti-PD-1 checkpoint blockade utilizes cellular mechanisms partially distinct from monotherapies. Proc Natl Acad Sci USA 116, 22699-22709 (2019).
84. T. R. Simpson et al., Fc-dependent depletion of tumor-infiltrating regulatory T cells co-defines the efficacy of anti-943 CTLA-4 therapy against melanoma. J Exp Med 210, 1695-1710 (2013).
85. N. Sharma, et al., TLR1/2 ligand enhances antitumor efficacy of CTLA-4 blockade by increasing intratumoral Treg depletion. Proc Natl Acad Sci USA 116, 10453-10462 (2019).
86. A. Musolino, et al., Immunoglobulin G fragment C receptor polymorphisms and clinical efficacy of trastuzumab-based therapy in patients with HER-2/neu-positive metastatic breast cancer. J Clin Oncol 26, 1789-1796 (2008).
87. R. J. Taylor, et al., FcgammaRIIIa polymorphisms and cetuximab induced cytotoxicity in squamous cell carcinoma of the head and neck. Cancer Immunol Immunother 58, 997-1006 (2009).
88. J. Pander, et al., Activation of tumor-promoting type 2 macrophages by EGFR-targeting antibody cetuximab. Clin Cancer Res 17, 5668-5673 (2011).
89. P. Bruhns, Properties of mouse and human IgG receptors and their contribution to disease models. Blood 119, 5640-5649 (2012).
90. A. J. Wisdom et al., Single cell analysis reveals distinct immune landscapes in transplant and primary sarcomas that determine response or resistance to immunotherapy. Nat Commun 11, 6410 (2020).
91. M. R. Crittenden, et al., Tumor cure by radiation therapy and checkpoint inhibitors depends on pre-existing immunity. Sci Rep 8, 7012 (2018).
92. M. A. Aznar, et al., Intratumoral Delivery of Immunotherapy-Act Locally, Think Globally. J Immunol 198, 31-39 (2017).
93. J. C. Jagodinsky, et al., The Promise of Combining Radiation Therapy with Immunotherapy. Int J Radiat Oncol Biol Phys, (2020).
94. D. Bauche, et al., LAG3(+) Regulatory T Cells Restrain Interleukin-23-Producing CX3CR1(+) Gut-Resident Macrophages during Group 3 Innate Lymphoid Cell-Driven Colitis. Immunity 49, 342-352 e345 (2018).
95. A. Assouvie, et al., Growing Murine Bone Marrow-Derived Macrophages. Methods Mol Biol 1784, 29-33 (2018).
96. H. M. Rice, et al., rM-CSF efficiently replaces L929 in generating mouse and rat bone marrow-derived macrophages for in vitro functional studies of immunity to intracellular bacteria. J Immunol Methods 477, 112693 (2020).
97. S. Manzanero, Generation of mouse bone marrow-derived macrophages. Methods Mol Biol 844, 177-181 (2012).

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

1. A method of treating cancer in a subject in need thereof, comprising:
a) administering a therapeutically effective amount of an adjuvant to a tumor of the subject, wherein the adjuvant is administered intratumorally;
b) administering a therapeutically effective amount of a radiotherapy and/or local ablative therapy to the tumor;
c) administering a therapeutically effective amount of an immune checkpoint inhibitor to the subject; and
d) inducing an immune response to the cancer.

2. The method of claim 1, wherein the adjuvant comprises a TLR4 agonist.

3. The method of claim 2, wherein the TLR4 agonist comprises one or more of monophosphoryl lipid A, monophosphoryl lipid A-504, monophosphoryl tri-acyl lipid A, monophosphoryl 3-deacyl lipid A, monophosphoryl tetra-acyl lipid A, monophosphoryl hexa-acyl lipid A, 3-deacyl, D-(+)-trehalose 6,6′-dibehenate, and dimethyldioctadecylammonium (bromide salt).

4. The method of claim 1, wherein the therapeutically effective amount of the adjuvant comprises about 20 μg to about 70 mg or about 0.5 to about 5 mg/kg.

5. The method of claim 1, wherein the radiotherapy comprises external beam radiation therapy (EBRT) and/or internal radiation therapy.

6. The method of claim 5, wherein the radiotherapy is EBRT.

7. The method of claim 5, wherein the internal radiation therapy comprises brachytherapy and/or radiopharmaceutical.

8. The method of claim 1, wherein the therapeutically effective amount of radiotherapy is about 2 to about 20 Gy.

9. The method of claim 1, wherein the therapeutically effective amount of radiotherapy is administered in a gradient dose of about 2 Gy/min.

10. The method of claim 1, wherein the local ablative therapy comprises radiofrequency ablation, microwave ablation, and/or cryoablation.

11. The method of claim 1, wherein the immune checkpoint inhibitor comprises one or more therapeutic agents that inhibit CTLA-4, PD-1, and/or PD-L1.

12. The method of claim 11, wherein the immune checkpoint inhibitor comprises an anti-CTLA-4 antibody.

13. A composition for treating cancer, comprising:
a) a therapeutically effective amount of an adjuvant;
b) a therapeutically effective amount of an immune checkpoint inhibitor; and
c) a pharmaceutically acceptable carrier or diluent.

14. The composition of claim 13, wherein the adjuvant comprises a TLR4 agonist.

15. The composition of claim 14, wherein the TLR4 agonist comprises one or more of monophosphoryl lipid A, monophosphoryl lipid A-504, monophosphoryl tri-acyl lipid A, monophosphoryl 3-deacyl lipid A, monophosphoryl tetra-acyl lipid A, monophosphoryl hexa-acyl lipid A, 3-deacyl, D-(+)-trehalose 6,6′-dibehenate, and dimethyldioctadecylammonium (bromide salt).

16. The composition of claim 13, wherein the immune checkpoint inhibitor comprises one or more therapeutic agents that inhibit CTLA-4, PD-1, and/or PD-L1.

17. The composition of claim 16, wherein the immune checkpoint inhibitor comprises an anti-CTLA-4 antibody.

18. A method of treating cancer in a subject in need thereof, comprising:
a) administering a therapeutically effective amount of a TLR4 agonist to a tumor of the subject, wherein the TLR4 agonist is administered intratumorally;
b) administering a therapeutically effective amount of EBRT to the tumor;
c) administering a therapeutically effective amount of an anti-CTLA-4 antibody to the subject; and
d) at least one of reducing tumor volume, increasing overall survival of the subject, and increasing complete response rate in the subject.

19. The method of claim 18, wherein the TLR4 agonist comprises one or more of monophosphoryl lipid A, monophosphoryl lipid A-504, monophosphoryl tri-acyl lipid A, monophosphoryl 3-deacyl lipid A, monophosphoryl tetra-acyl lipid A, monophosphoryl hexa-acyl lipid A, 3-deacyl, D-(+)-trehalose 6,6′-dibehenate, and dimethyldioctadecylammonium (bromide salt).

20. The method of claim 19, wherein the therapeutically effective amount of the TLR4 agonist comprises about 0.5 to about 5 mg/kg.

21. The method of claim 18, wherein the therapeutically effective amount of EBRT is about 2 to about 20 Gy administered in a gradient dose of about 2 Gy/min.

22. The method of claim 18, wherein the therapeutically effective amount of the anti-CTLA-4 antibody is about 10 mg/kg.

23. (canceled)

24. The method of claim 1, wherein the cancer is melanoma or prostate cancer.

25. The method of claim 1 further comprising:
e) increasing production of Th1-associated, IgG2c anti-tumor antibodies associated with the tumor.

26. The method of claim 18 further comprising:
e) inducing a systemic anti-tumor immune response.
